(19) United States
(12) Patent Application Publication
(10) Pub. No.: US 2010/0166695 A1
(43) Pub. Date: Jul. 1, 2010

(54) Multivalent Heterobifunctional Polymers And Methods Of Their Use

(75) Inventors: David R. Bundle, Edmonton (CA); Pavel kitov, Edmonton (CA)

Correspondence Address: EDELL, SHAPIRO & FINNAN, LLC, 1901 RESEARCH BLVD., SUITE 400, ROCKVILLE, MD, 20850-3164, US

(73) Assignee: THE GOVERNORS OF THE UNIVERSITY OF ALBERTA, Edmonton, AL (CA)

(21) Appl. No.: 12/530,814

(22) PCT Filed: Mar. 20, 2008
(86) PCT No.: PCT/CA08/00531

§ 371 Date: Mar. 12, 2010

Related U.S. Application Data
(60) Provisional application No. 60/896,878, filed on Mar. 23, 2007.

(51) Int. Cl. A61K 31/70; C07H 15/08; C08B 37/02; C08F 251/00; A61K 31/74

(52) U.S. Cl. 424/78.18; 536/18.3; 536/51; 536/53; 514/25; 514/27; 527/312; 527/314; 424/78.17

(57) ABSTRACT

Designed herein are multivalent heterobifunctional polymers for binding to a biological target exhibiting biological activity and to an effector template which can affect the biological activity of the biological target or detect the presence of the biological target. The polymers comprise a plurality of pre-arranged heterobifunctional ligands connected thereto, and each heterobifunctional ligand comprises a first functionality capable of binding to the biological target, and a second functionality capable of binding to the effector template. The heterobifunctional ligands are pre-arranged on the polymer so as to form a ternary complex between the polymer, the biological target and the effector template. The polymers, methods and compositions described herein provide an approach for the design and production of new therapeutic agents as well as agents useful in a variety of non-therapeutic applications.

### CROSS REFERENCE TO RELATED APPLICATION

This application claims the benefit of U.S. Provisional Patent Application No. 60/896,878, filed Mar. 23, 2007, which is incorporated herein in its entirety.

### FIELD OF THE INVENTION

The present invention relates to novel multivalent polymers containing heterobifunctional ligands, methods for their synthesis, compositions thereof, and therapeutic and non-therapeutic applications thereof.

### BACKGROUND

Specific interactions between different biological entities are central to many biological processes which include, but are not limited to, cell-cell communication, cellular responses to the environment, cell differentiation, cell proliferation, cell migration, signal transduction, metabolic processes, apoptosis and immune responses. In many cases, these important processes and responses are mediated through the interaction of specific ligands with specific targets. These ligands and targets can include, but are not limited to, proteins, DNA, RNA, carbohydrates, lipids, and cells.

The importance of interactions between various ligands and their targets is underscored by the large list of disorders that are caused by the breakdown of these interactions or by the breakdown of the regulation of these interactions. As a greater understanding of these specific interactions is gained, it is hoped that they can be controlled and harnessed for both therapeutic and non-therapeutic applications.

Recently, much research has focused on the design and synthesis of a novel class of ligands, which can interact with various biological entities and assist in the treatment of infectious and non-infectious diseases. This novel class of ligands includes homo- and heterobifunctional ligands. These ligands bind to targeted biological cells or molecules and also bind to an endogenous protein or antibody such that the formation of the ternary complex promotes the elimination of the targeted cell or molecule. Such bifunctional ligands provide a novel approach for removing not only bacterial and viral particles and virally infected cells, but also unwanted cells, proteins, antibodies, and other biological molecules involved in a variety of debilitating diseases. This supramolecular protein aggregation offers exciting opportunities for both therapeutic and non-therapeutic applications.

Bifunctional ligands known to date typically have at least two head groups for binding to their respective targets. In homobifunctional ligands, the two head groups are similar or identical, are attached to one another directly or via a linker, and promote the aggregation of like targets (Pepys, M. B. WO 03/013508; Bundle et al. US Patent Application No. 2007/0042936).

In heterobifunctional ligands, the two head groups are unique, are attached to one another directly or via a linker, and promote the specific aggregation of dissimilar targets (Shokat, K. M., and Schultz, P. G. 1991 J. Am. Chem. Soc. 113:1861-1862; Pepys, M. B. WO 03/013508; Mullis, K. B. U.S. patent application Ser. No. 10/178,046 and U.S. patent application Ser. No. 10/696,770; Liu, J. et al., 2005 J. Am. Soc. 127:2044-2045). Liu et al. discloses a heterobifunctional ligand that binds both to cholera toxin and to human serum amyloid P component (SAP), an endogenous protein of the innate immune system. SAP directs removal of the ternary complex from circulation via the liver. Inhibition of the cholera toxin was found to be three orders of magnitude greater in the ternary complex than that seen in the binary complex of just the heterobifunctional ligand and the toxin. The increased inhibition resulting from formation of the ternary complex is also seen in the heterobifunctional ligand reported by Solomon et al. (2005 Organic Letters 7:4369-4372) where the heterobifunctional ligand mediates the specific aggregation of the E. coli Shiga-like toxin with SAP.

However, currently known homo- and heterobifunctional ligands have many serious disadvantages and limitations. First, many of the currently known ligands are rapidly cleared upon administration. This rapid clearance seriously undermines the potential therapeutic benefits of these ligands. Second, the binding of many of the homo- and heterobifunctional ligands can be compromised because of entropy costs. In many cases, currently known bifunctional ligands enjoy a fairly high level of flexibility in their unbound form, and must suffer high loss of this flexibility upon binding their target. The loss of flexibility is energetically unfavourable and can seriously impede binding efficiency. Third, heterobifunctional ligands do not take advantage of valency. Many studies have now shown that the valency of binding may be an important feature of specific interactions at the heart of important biological processes. In fact, it is now believed that many important biological processes result from the simultaneous interaction of multiple ligands with multiple binding sites of the target receptors. Fourth, use of heterobifunctional ligands to form ternary complexes may be limited depending on the concentration of one of the targets. This may compromise the use of these ligands in therapeutic applications. Fifth, the identity of the head groups on the ligands may be restricted to only those that show strong binding to the target.

Recently, various ligands have been covalently anchored onto a polymeric chain in an attempt to avoid some of these problems. Examples of polymeric ligands where two different head groups are individually and independently anchored at various locations onto a polymeric chain are known (see FIG. 1) (Krishnamurthy, V. M. et al. 2006, Biomaterials 27:3663-3674; Krishnamurthy, V. M. et al. WO 2007/016556; Whitesides, G. et al. WO 98/46270; Kiessling et al. US 2003/0125262). However, these polymers also have many serious disadvantages and limitations. First, and foremost, the binding strength of the polymeric ligand to each receptor is not influenced by whether or not the other receptor is present. While currently known polymeric ligands are able to attract two different biological receptors to one another, the independent anchoring of the two different ligands on the polymer does not allow the ligands to take advantage of entropy savings due to formation of defined supramolecular complexes. This can severely impede the therapeutic and non-therapeutic usefulness of these polymers.

Consequently, the need has arisen for multivalent polymers that can be efficiently used to form ternary complexes in vitro and in vivo, while avoiding some of the problems listed above.

### SUMMARY

In accordance with a broad aspect of this invention, there is provided a multivalent heterobifunctional polymer for binding to a biological target exhibiting biological activity and to an effector template which can affect the biological activity of the biological target or detect the presence of the biological target, where the polymer comprises a plurality of pre-arranged heterobifunctional ligands connected thereto, the heterobifunctional ligands comprising a first functionality capable of binding to the biological target, and a second functionality capable of binding to the effector template, wherein the heterobifunctional ligands are pre-arranged on the polymer so as to form a ternary complex between the polymer, the biological target and the effector template. The first functionality and the second functionality can be selected from the group consisting of an amino acid, a peptide, a derivatized peptide, a monosaccharide, an oligosaccharide, a vitamin, a nucleotide, a nucleotide analog, a polynucleotide, a polynucleotide analog, a cell nutrient, an antigenic determinant, a small drug-like compound, a hapten, an antibody or antibody fragment, a cell surface receptor, and combinations and analogs thereof. The biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, an archaebacteria, a fungus, a viral particle, a bacterial toxin, viral lectins, a cancer cell, B cells, and combinations and analogs thereof. The effector template can be selected from the group consisting of a multivalent receptor, a multivalent protein, a protein, a peptide, a derivatized peptide, an antibody, a membrane-bound receptor, and combinations and analogs thereof. The polymer may be selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers. In one embodiment, the first functionality and the second functionality are attached to a common atom, wherein the common atom is attached directly or via a linker to or into the polymer backbone. In another embodiment, the first functionality and the second functionality are directly, or via an optional linker, attached to one another, and either the first functionality or the second functionality is attached directly or via a linker to or into the polymer backbone.

In accordance with another broad aspect of this invention, there is provided a multivalent heterobifunctional polymer for binding to a biological target exhibiting biological activity and to an effector template which can affect the biological activity of the biological target or detect the presence of the biological target, the polymer having the formula:

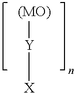

wherein “X” represents a polymeric backbone of the multivalent polymer; “MO” represents a heterobifunctional ligand, wherein “M” represents a first functionality capable of binding to the biological target and “O” represents a second functionality capable of binding to the effector template; “Y” represents an optional linker that connects “MO” to or into the polymeric backbone; and “n” represents an integer selected such that a sufficient number of heterobifunctional ligands are presented in the polymer for an intended use. In one embodiment, “n” is selected such that the number of heterobifunctional ligands on the polymer is the same as or greater than the number of receptors on the biological target and the effector template, whichever is greater. The polymer may be selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers. In one embodiment, “M” is connected to or into the polymeric backbone. In one embodiment, “M” is connected to or into the polymeric backbone through linker “Y”. In one embodiment, “O” is connected to or into the polymeric backbone. In one embodiment, “O” is connected to or into the polymeric backbone through linker “Y”. In one embodiment, “M” and “O” are connected to each other by a linker.

In accordance with another broad aspect of this invention, there is provided a multivalent heterobifunctional polymer for binding to a biological target exhibiting biological activity and to an effector template which can affect the biological activity of the biological target or detect the presence of the biological target, the polymer having the formula:

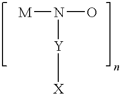

wherein “X” represents a polymeric backbone of the multivalent polymer; “M-N-O” represents a heterobifunctional ligand, wherein “M” represents a first functionality capable of binding to the biological target, “O” represents a second functionality capable of binding to the effector template, and “N” represents a linker connecting “M” and “N”; “Y” represents an optional linker that connects the heterobifunctional ligand to or into the polymeric backbone; and “n” represents an integer selected such that a sufficient number of heterobifunctional ligands are presented in the polymer for an intended use. In one embodiment, “n” is selected such that the number of heterobifunctional ligands on the polymer is the same as the number of receptors on the biological target and the effector template, whichever is greater. The polymer may be selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers.

In accordance with another broad aspect of this invention, there is provided a method for affecting the biological activity of a biological target in a biological system, the method comprising introducing into the biological system a multivalent heterobifunctional polymer for binding to the biological target exhibiting biological activity and to an effector template which can affect the biological activity of the biological target, the polymer comprising a plurality of pre-arranged heterobifunctional ligands, the heterobifunctional ligands comprising a first functionality capable of binding to the biological target, and a second functionality capable of binding to the effector template, wherein the heterobifunctional ligands are pre-arranged on the polymer so as to form a ternary complex between the polymer, the biological target and the effector template. The first functionality and the second functionality can be selected from the group consisting of an amino acid, a peptide, a derivatized peptide, a monosaccharide, an oligosaccharide, a vitamin, a nucleotide, a nucleotide analog, a polynucleotide, a polynucleotide analog, a cell nutrient, an antigenic determinant, a small drug-like compound, a hapten, an antibody or antibody fragment, a cell surface receptor, and combinations and analogs thereof. The biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a viral particle, a bacterial toxin, viral lectins, a cancer cell, B cells, a unicellular parasite, an archaebacteria, a fungus, and combinations and analogs thereof. The effector template can be selected from the group consisting of a multivalent receptor, a multivalent protein, a protein, a peptide, a derivatized peptide, an antibody, a membrane-bound receptor, and combinations and analogs thereof. In one embodiment, the first functionality and the second functionality are attached to a common atom, wherein the common atom is attached directly or via a linker to or into the polymer backbone. In one embodiment, the first functionality and the second functionality are directly, or via an optional linker, attached to one another, and either the first functionality or the second functionality is attached directly or via a linker to or into the polymer backbone.

In accordance with another broad aspect of this invention, there is provided a method for detecting the presence of a biological target in a biological system the method comprising introducing into the biological system a multivalent heterobifunctional polymer for binding to the biological target exhibiting biological activity and to an effector template which can detect the presence of the biological target, the polymer comprising a plurality of pre-arranged heterobifunctional ligands, the heterobifunctional ligands comprising a first functionality capable of binding to the biological target, and a second functionality capable of binding to the effector template, wherein the heterobifunctional ligands are pre-arranged on the polymer so as to form a ternary complex between the polymer, the biological target and the effector template. The first functionality and the second functionality can be selected from the group consisting of an amino acid, a peptide, a derivatized peptide, a monosaccharide, an oligosaccharide, a nucleotide, a nucleotide analog, a polynucleotide, a polynucleotide analog, a vitamin, a cell nutrient, an antigenic determinant, a small drug-like compound, a hapten, an antibody or antibody fragment, a cell surface receptor, and combinations and analogs thereof. The biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, a fungus, a viral particle, a bacterial toxin, viral lectins, a cancer cell, B cells, and combinations and analogs thereof. The effector template can be selected from the group consisting of a multivalent receptor, a multivalent protein, a protein, a peptide, a derivatized peptide, an antibody, a membrane-bound receptor, and combinations and analogs thereof. In one embodiment, the first functionality and the second functionality are attached to a common atom, wherein the common atom is attached directly or via a linker to or into the polymer backbone. In one embodiment, the first functionality and the second functionality are directly, or via an optional linker, attached to one another, and either the first functionality or the second functionality is attached directly or via a linker to or into the polymer backbone.

In accordance with another broad aspect of this invention, there is provided a pharmaceutical composition for affecting the biological activity of a biological target in a biological system, the composition comprising a multivalent heterobifunctional polymer for binding to the biological target exhibiting biological activity and to an effector template which can affect the biological activity of the biological target, the polymer comprising a plurality of pre-arranged heterobifunctional ligands, the heterobifunctional ligands comprising a first functionality capable of binding to the biological target, and a second functionality capable of binding to the effector template, wherein the heterobifunctional ligands are pre-arranged on the polymer so as to form a ternary complex between the polymer, the biological target and the effector template; and a pharmaceutically acceptable excipient. The first functionality and the second functionality can be selected from the group consisting of an amino acid, a peptide, a derivatized peptide, a monosaccharide, an oligosaccharide, a nucleotide, a nucleotide analog, a polynucleotide, a polynucleotide analog, a vitamin, a cell nutrient, an antigenic determinant, a small drug-like compound, a hapten, an antibody or antibody fragment, a cell surface receptor, and combinations and analogs thereof. The biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, a fungus, a viral particle, a bacterial toxin, viral lectins, a cancer cell, B cells, and combinations and analogs thereof. The effector template can be selected from the group consisting of a multivalent receptor, a multivalent protein, a protein, a peptide, a derivatized peptide, an antibody, a membrane-bound receptor, and combinations and analogs thereof. In one embodiment, the first functionality and the second functionality are attached to a common atom, wherein the common atom is attached directly or via a linker to or into the polymer backbone. In one embodiment, the first functionality and the second functionality are directly attached to one another, and either the first functionality or the second functionality is attached directly or via a linker to or into the polymer backbone.

In accordance with another broad aspect of this invention, there is provided a method for pre-arranging a plurality of heterobifunctional ligands on a multivalent heterobifunctional polymer, the heterobifunctional ligands being connected at different connection points on the polymer and comprising a first functionality for binding a biological target and a second functionality for binding an effector template to form a ternary complex, the method comprising the steps of aligning molecular representations of the biological target and the effector template using molecular modeling or visualization software; measuring the average distance separating two similar or identical adjacent binding sites on the effector template; measuring the average distance separating two similar or identical adjacent binding sites on the biological template; and measuring the average distance separating the first functionality and the nearest second functionality when bound to the biological target and the effector template, wherein the heterobifunctional ligands are pre-arranged on the multivalent heterobifunctional polymer so that the average distance separating the first functionality and the second functionality is minimized without introducing steric clashes between the biological target and the effector template in the ternary complex. In one embodiment, the topology of the binding sites of the biological target and the effector template are similar or identical. In one embodiment, the distance separating the first functionality and the nearest second functionality when bound to the biological target and the effector template is optimized by varying the length of one or more than one linker connecting the first functionality to the second functionality. In one embodiment, the average distance separating the first functionality and the nearest second functionality when bound to the biological target and the effector template is equal to or smaller than the length of an optional linker connecting the first functionality to the second functionality. In one embodiment, the average distance separating the first functionality and the nearest second functionality when bound to the biological target and the effector template is optimized by varying the length of a linker connected the first functionality and the second functionality. In one embodiment, heterobifunctional ligands are pre-arranged on the multivalent heterobifunctional polymer so that the sum of the average distance separating the connection point of one heterobifunctional ligand from the connection point of an adjacent heterobifunctional ligand, two times the length of an optional linker attaching the heterobifunctional ligands to or into the polymer, and the length of an optional linker connecting the first functionality to the second functionality is greater than the larger of the average distance separating two similar or identical adjacent binding sites on the effector template or the average distance separating two similar or identical adjacent binding sites on the biological template; and the length of the linker connecting the first functionality to the second functionality is less than the sum of the average distance separating the connection point of one heterobifunctional ligand from the connection point of the adjacent ligand, the length of the linker connecting the first functionality to the second functionality and two times the length of the linker attached the heterobifunctional ligands to or into the polymer.

In accordance with another broad aspect of this invention, there is provided the use of a multivalent heterobifunctional polymer of the present invention for a therapeutic application, wherein the therapeutic application is the treatment of a disease selected from the group consisting of cancer, a bacterial infection, a viral infection, a parasitic infection, a fungal infection, an autoimmune disease, hereditary and acquired metabolic disorders, and combinations thereof.

In accordance with another broad aspect of this invention, there is provided the use of a multivalent heterobifunctional polymer of the present invention for a non-therapeutic application, wherein the non-therapeutic application is selected from the group consisting of diagnostics, the detection of bacterial toxins in groundwater, the detection of antibodies in blood, the detection of cancer cells, and the imaging of tumors.

### BRIEF DESCRIPTION OF THE DRAWINGS

The present invention, both as to its organization and manner of operation, may best be understood by reference to the following description, and the accompanying drawings of various embodiments wherein like numerals are used throughout the several views, and in which:

FIG. 1 is a schematic diagram of a polymer of the prior art.

FIG. 2 is a schematic diagram of the formation of a ternary complex between a biological target, an effector template and a multivalent polymer of the present invention.

FIG. 3 is a schematic diagram of one embodiment of a multivalent polymer of the present invention, wherein each heterobifunctional ligand comprises a first functionality and a second functionality. In this embodiment, each functionality is attached to a common atom through a linker.

FIG. 4 is a schematic diagram of one embodiment of a multivalent polymer of the present invention, wherein each heterobifunctional ligand comprises a first functionality and a second functionality. In this embodiment, each functionality is attached to the other via an optional linker, but without the use of a common atom.

FIGS. 5A and 5B are schematic diagrams illustrating some of the principles of design of a multivalent polymer in one embodiment of the present invention. Multivalent polymers of the present invention can be designed using molecular modeling based on available structural information or homology modeling of proteins. The molecular dimensions of the construct are optimized to match molecular dimensions of the biological target and the effector template binding sites.

FIGS. 6A and 6B schematically describe the ELISA inhibition protocol used for to measure the inhibition of Stx1 by heterobifunctional polymers. FIG. 6A is a schematic representation of the structure of the synthetic P^{k}-trisaccharide attached to a C16 aglycon (16-mercaptohexadecanyl glycoside). FIG. 6B describes the ELISA inhibition assay. Wells were coated with synthetic P^{k}-trisaccharide attached to a C16 aglycon (16-mercaptohexadecanyl glycoside), then inhibitors were co-incubated with Stx1 (4 ng/mL) with and without SAP (20 μg/mL). Toxin bound to the plate was detected using rabbit anti-Stx1 polyclonal sera, followed by goat anti-rabbit horse radish peroxidase second antibody. Color was then developed with tetramethylbenzidine substrate.

FIG. 7 is a schematic diagram illustrating the structure of prior art polymer PPM and a polymer of the present invention, PPI.

FIG. 8 compares the Ca²⁺-dependent inhibition of binding to P^{k}-16-mercaptohexadecanyl glycoside ELISA plates (overnight coating at 10 μg/mL) by Shiga toxin type 1 (Stx1, 4 ng/mL) in the presence or absence of serum amyloid P (20 μg/mL) by prior art polymer PPM and polymer of the present invention PPI. Series 1: PPM in the presence of SAP. Series 2: PPM in the absence of SAP. Series 3: PPI in the presence of SAP. Series 4: PPI in the absence of SAP. As can be seen, the activity of PPM is not affected by the presence of SAP. Moreover, the inhibitory activity of PPI is 6000-fold higher in the presence of SAP than in the absence of SAP.

FIG. 9 compares the Ca²⁺-dependent inhibition of binding to P^{k}-16-mercaptohexadecanyl glycoside ELISA plates (overnight coating at 10 μg/mL) by Shiga toxin type 1 (Stx1, 4 ng/mL) in the presence of serum amyloid P (20 μg/mL) by polymers of the present invention HPMA-B1 and HPMA-B2. Open boxes (series 1) correspond to data collected using HPMA-B1 in the presence of SAP. Closed boxes (series 2) correspond to data collected using HPMA-B2 in the presence of SAP. HPMA-B2 has a dramatically smaller IC₅₀ value (0.065 μg/mL) than HPMA-B1 (12.8 μg/mL), which demonstrates the importance of optimization in the pre-arrangement of the heterobifunctional ligands on the polymer.

FIG. 10 represents the results of the Vero cytotoxicity neutralization assay. Stx1 (at LD₁₀₀ of approximately 2.91 ng/mL) was added to confluent cell culture in a mixture with serially diluted inhibitors and SAP (10 μg/mL) in Medium Eagle Medium (MEM) supplemented with fetal bovine serum in an atmosphere of 5% CO₂/95% air. Series 1: Results of the Vero cytotoxicity neutralization assay performed in the presence of EPI-156, a multivalent heterobifunctional ligand of the present invention. Series 2: Results of the Vero cytotoxicity neutralization assay performed in the presence of BAIT2, a BAIT2 is univalent analog of EPI-156, which is known in the prior art. Series 3: Results of the Very cytotoxicity neutralization assay performed in the presence of DAISY 1/8, a homodecameric P^{k}-trisaccharide containing a radially symmetric dendrimer, known in the prior art.

FIG. 11 is a schematic diagram of the structures of BAIT2, EPI-156 and EPI-153. Here, EPI-156 is shown in a modified form, with the addition of a tyrosine residue to allow for iodination.

FIG. 12 represents data obtained from the mouse intoxication model, which measures mouse survival following administration of Stx1 and various inhibitory polymers of the prior art and of the present invention. HuSAP mice were injected intravenously via the tail vein with a lethal dose (LD₅₀) of Stx1 and they were monitored every 4 hours for signs of shigatoxemia. Mice displaying signs of shigatoxemia were euthanized. Series 1 represents the percentage of mouse survival following administration of DAISY 1/8 (a P^{k}-containing dendrimer of the prior art) at 500 μg/mouse. Series 2 represents the percentage of mouse survival following administration of EPI-156 (a polymer of the present invention) at 50 μg/mouse. Series 3 represents the percentage of mouse survival following administration of EPI-153 (an inactive truncated-ligand analog of EPI-156) at 50 μg/mouse and HuSAP at 600 μg/mouse. Series 4 represents the percentage of mouse survival following administration of EPI-156 at 50 μg/mouse and HuSAP at 600 μg/mouse. Series 5 represents the percentage of mouse survival following administration of BAIT2 at 2 mg/mouse. As can be observed, intravenous administration of EP-156 alone, a polymer of the present invention, is sufficient to protect mice from the toxic effects of Stx1 through the promotion of the formation of ternary complexes between Stx1, EP-156 and HuSAP, expressed in the transgenic mice.

FIGS. 13A and 13B represent the organ distribution of radioactively labeled EPI-156 (EPI-156-¹²⁵I) and Shiga toxin (Stx1-¹²⁵I) measured 4 hours after post-intravenous injection into transgenic mice expressing human SAP (HuSAP mice). In FIG. 13A, HuSAP mice received 900 ng of EPI-156-¹²⁵I (1.14×10⁷ CPM/μg) via tail vein injection. Solid bars represent the organ distribution of a mixture of EPI-156-¹²⁵I and HuSAP, whereas open bars represent the organ distribution of a mixture of EPI-156-¹²⁵I, HuSAP and Stx1. From FIG. 13A, it can be observed that the heterobifunctional polymeric ligand is directed to the liver. In FIG. 13B, mice received 20 ng/g of Stx1-¹²⁵I (4.81×10⁶ CPM/μg) via tail vein injection. Solid bars represent the organ distribution of a mixture of Stx1 and HuSAP, whereas open bars represent the organ distribution of a mixture of Stx1, HuSAP and non-labeled EPI-156. From FIG. 13B, it can be observed that, in the presence of multivalent heterobifunctional polymers of the present invention, the toxin Stx1 is directed to the liver instead of being directed to the kidneys and lungs, which is observed in the absence of the polymers. This could explain the protective action of these polymers.

### DETAILED DESCRIPTION OF THE INVENTION

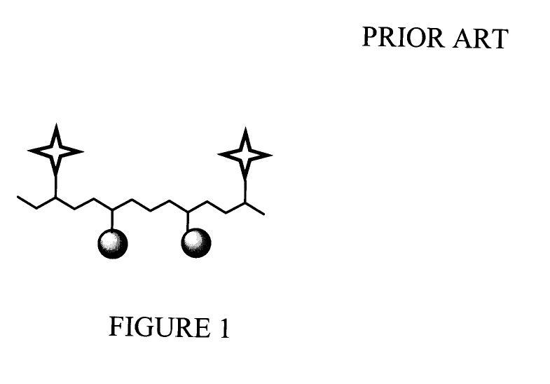

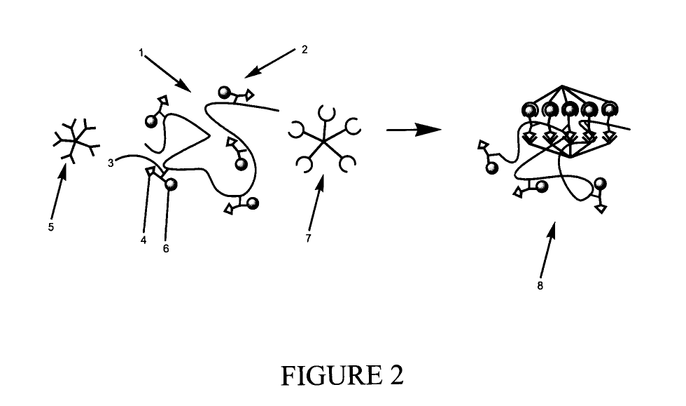

The present invention relates to the discovery of multivalent heterobifunctional polymers, methods for their synthesis, compositions thereof, and therapeutic and non-therapeutic applications thereof. A distinctive feature of the multivalent polymers of the present invention is the presence of a plurality of heterobifunctional ligands connected thereto, where each heterobifunctional ligand comprises two different binding functionalities (see FIG. 2). Each binding functionality on the heterobifunctional ligand is pre-arranged so that each can engage a different biological entity. This differs from polymeric ligands of the prior art that present two independent unifunctional head groups connected at different locations on the polymer (see FIG. 1). Surprisingly, the inventors have found that the pre-arrangement of the two binding functionalities of the heterobifunctional ligands on the multivalent polymers of the present invention allows for the formation of ternary complexes whose stability cannot be replicated by the use of currently known polymers (see FIG. 1), which have two independent unifunctional head groups attached at different locations on the polymer. Without wishing to be bound by theory, through the use of the multivalent heterobifunctional polymers of the present invention, both multivalency and supramolecular effects operate by reclaiming a portion of the binding entropy that is lost upon formation of a complex between the target receptor and several copies of a univalent ligand. These multivalent polymers are particularly advantageous since multivalency and supramolecular effects can supplement one another and allow for substantial gains in binding free energy. This can be particularly advantageous in that the binding to one biological entity can serve to strengthen the binding of the polymer to another biological entity.

As illustrated in FIG. 2, a multivalent polymer 1 of the present invention comprises a plurality of heterobifunctional ligands 2 that are connected to multivalent polymer 1 through an optional linker 3. Each heterobifunctional ligand 2 comprises a first functionality 4 that can bind to a biological target 5, and a second functionality 6 that can bind to an effector template 7, so that a ternary complex 8 can be formed. Without wishing to be bound by theory, formation of ternary complex 8 through the binding of multivalent polymer 1 to biological target 5 and effector template 7 promotes the detection, inhibition, elimination and/or clearance of one biological entity by the other.

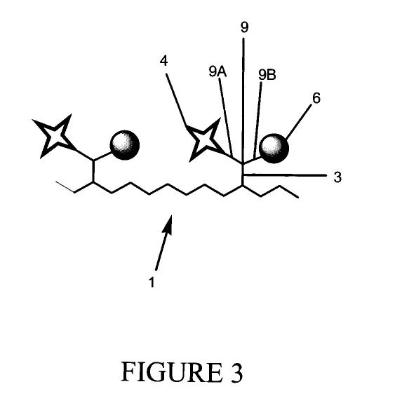

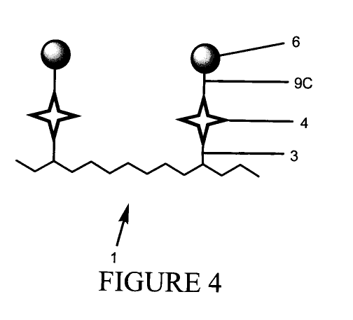

As illustrated in FIGS. 3 and 4, heterobifunctional ligands can display first functionality 4 and second functionality 6 in a variety of formats. As will be discussed below, the format chosen will depend on the intended use and function. In one embodiment shown in FIG. 3, first functionality 4 and second functionality 6 of heterobifunctional ligand 2 are connected to a common atom 9, via linkers 9A and 9B, respectively. Common atom 9 can be connected via optional linker 3 to multivalent polymer 1. Of course, as will be understood by one of skill in the art, common atom 9 may also be directly connected to multivalent polymer 1 without the aid of linker 3 (not shown).

In another embodiment shown in FIG. 4, first functionality 4 and second functionality 6 can be connected to one another through optional linker 9C without the use of common atom 9, and either first functionality 4 or second functionality 6 can be attached via optional linker 3 to multivalent polymer 1. Of course, as will be understood by one of skill in the art, the resulting complex of first functionality 4 and second functionality 6 may also be directly connected to multivalent polymer 1 without the aid of linker 3 (not shown).

As discussed above, the pre-arrangement of first functionality 4 and second functionality 6 of heterobifunctional ligands 2 on multivalent polymer 1 is important in order to achieve high binding efficiency. The pre-arrangement of the two functionalities of the heterobifunctional ligands on the polymer will be dependent on biological target 5 and effector template 7. In one embodiment, biological target 5 and effector template 7 are very similar or topologically identical in terms of the relative positions and arrangement of their binding site(s). This similarity can be used advantageously to pre-arrange first functionality 4 and second functionality 6 of heterobifunctional ligands 2 connected to the polymers of the present invention. Using these similarities, a polymer of the present invention can be designed so that the formation of ternary complexes is maximized. One of skill in the art will appreciate that optimization of the formation of ternary complexes through pre-arrangement of first functionality 4 and second functionality 6 may involve varying the lengths of any linkers described above that may be used, as well as varying the average distance separating the adjacent connection points of the heterobifunctional ligands on the polymer. The lengths of the linkers and the average distance separating the adjacent connection points of the heterobifunctional ligands can be easily modified using chemical synthesis techniques known in the art. One of skill in the art will also appreciate that the biological activity of the resulting polymer may be determined using a wide variety of assays, whose identity will depend on the identities of biological target 5 and effector template 7.

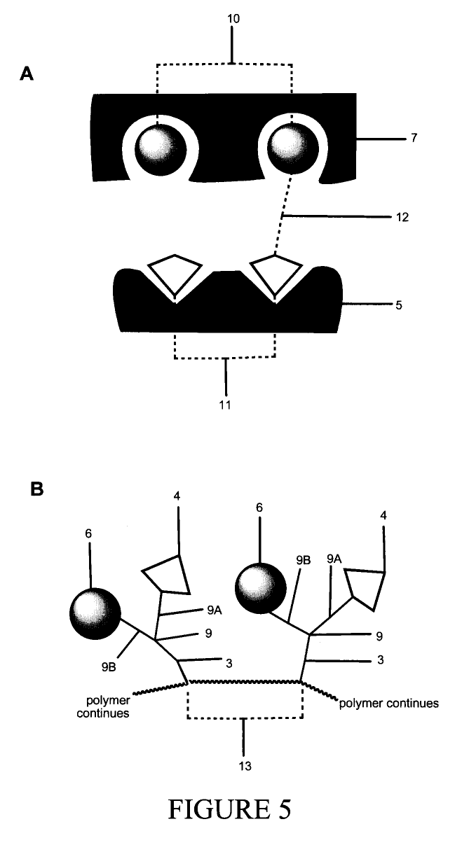

In another embodiment, when structural data are available for biological target 5 and/or effector template 7, the spatial pre-arrangement of first functionality 4 and second functionality 6 can be facilitated by studying the known or predicted structure and/or binding site(s) of biological target 5 and effector template 7. Structure may be predicted using a wide variety of molecular modeling tools known in the art. As shown in FIG. 5A, when designing a multivalent polymer of the present invention in light of known structural data for biological target 5 and/or effector template 7, at least the following three distances should be considered: the average distance 10 separating two similar or identical adjacent binding sites on effector template 7, the average distance 11 separating two similar or identical adjacent binding sites on biological target 5, and the average distance 12 separating first functionality 4 and the nearest second functionality 6 when they are bound to their respective binding sites. Preferably, distance 12 is measured when biological target 5 and effector template 7 are aligned so that this distance is minimized. Of course, as will be appreciated by one of skill in the art, molecular representations of biological target 5 and effector template 7 may be aligned using molecular modeling or visualization software known in the art. Distance 12 should be minimal without imposing clashes between biological target 5 and effector template 7 in the resulting ternary complex. As will be appreciated by one of skill in the art, all three distances can be determined using various molecular modeling or visualization software currently known in the art.

In one embodiment, when structural data are available for biological template 5 and effector template 7, distances 10, 11 and 12 can be used as a general guide in order to pre-arrange heterobifunctional ligands onto multivalent polymer 1 to promote formation of stable ternary complexes. In deciding how to spatially arrange first functionality 4 and second functionality 6 in relation to one another, the sum of the length of linkers 9A and 9B or the length of linker 9C should be greater or equal to distance 12. The distance 13 that separates one heterobifunctional ligand from another as well as the length of linker 3 are also needed to design multivalent polymer 1 (see FIG. 5B). Preferably, the sum of distance 13, two times the length of linker 3 and two times the length of linker 9A should be greater than distance 10 or 11, whichever is greater. In one embodiment, the sum of distance 13, two times the length of linker 3 and the length of linker 9C should be greater than distance 10 or 11, whichever is greater.

Other relationships can also be optionally used to help in the design and pre-arrangement of the two functionalities in the heterobifunctional ligands when structural data are available. Preferably, the sum of the length of linkers 9A and 9B should be less than the sum of the length of distance 13, two times the length of linker 9A and two times the length of linker 3. Moreover, the sum of the length of linkers 9A and 9B should be less than the sum of the length of distance 13, two times the length of linker 9B and two times the length of linker 3. In another embodiment, the length of linker 9C should be less than the sum of the length of distance 13, the length of linker 9C and two times the length of linker 3.

When measuring the various average distances described above, it is preferable to assume that any fragment or linker that joins two functionalities and/or ligands is in an extended conformation, where the reference points are the centre of mass of each functionality and/or ligand. As one of skill in the art will appreciate, an extended conformation of a flexible molecule is the conformation that provides the greatest possible distance between two reference points. As discussed above, these distances can be estimated through the use of molecular models, which can take into account appropriate covalent bond lengths and angles. In addition, due to the essentially random nature of co-polymerization reactions and reactions used for modification of a pre-formed polymer, the value of distance 13 can only be estimated as an average in a statistical sense via the rate of incorporation of heterobifunctional ligands into a polymer. For instance, when the ligand-to-repeat unit ratio is 1:20, distance 13 is assumed to be equal to the end-to-end length of the polymeric chain, which consists of 20 repeat units.

When using known structural data to pre-arrange heterobifunctional ligands 2, the four different constraints listed above are generally required since the probability of finding flexible polymeric molecules in an extended conformation is generally very low. Because of this, further optimization of the structure of the heterobifunctional multivalent ligands is generally required to promote the formation of stable ternary complexes since longer than merely sufficient length linkers are generally required to increase the probability of finding binding functionalities at distances 10, 11 and 12. As mentioned above, the lengths of the linkers and the distance separating the adjacent connection points of the heterobifunctional ligands can be easily optimized to increase the probability of forming stable ternary complexes. Optimization can be carried out using a wide variety of techniques known in the art (for example, which are not meant to be limiting, Kitov, P. I., et al. 2002, J. Am. Chem. Soc. 125:3284-3294; Mammen, M., et al. 1998, J. Org. Chem. 63:3168-3175; Gargano, J. M., et al. 2001, J. Am. Chem. Soc. 123:12909-12910).

In the four constraints described above, at least distance 13 should have a positive, non-zero value. However, as discussed above and illustrated in FIG. 4, the lengths of linkers 9A, 9B and/or 9C may be zero. As will be shown below, the inventors have found that first functionality 4 and second functionality 6 may be joined together without the use of linkers, while creating entropically efficient binding moieties.

In one embodiment, multivalent polymer 1 of the present invention can be represented as follows:

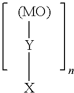

In certain embodiments, multivalent polymer 1 of the present invention can be represented by the following structure:

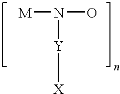

wherein “X” represents the polymeric backbone of multivalent polymer 1, “M” represents first functionality 4, “O” represents second functionality 6, “N” represents is either common atom 9, an optional linker, or a bond which can be used to connect “M” and “O” directly as discussed above, “Y” represents optional linker 3, and “n” represents an integer, selected such that a sufficient number of heterobifunctional ligands are presented in the polymer for the intended use. Preferably, the value of “n” should be the same as the number of binding sites in the targeted receptors.

### First Functionality and Second Functionality

The identity of biological target 5 and effector template 7 and, consequently, the identity of first functionality 4 and second functionality 6, can vary widely depending on the intended application. In one embodiment, biological target 5 is an entity mediating a disease, whereas effector template 7 is an entity capable of affecting the biological activity of the biological target or allowing for the detection of the biological target.

The term “biological activity” refers to any deleterious activity exerted by biological target 5. Without wishing to be bound by theory, second functionality 6 that can engage effector template 7 can affect the exhibited biological activity of biological target 5 through a variety of mechanisms which include, but are not limited to, localization of resulting ternary complex 8 to a specific organ such as the liver, promotion of the association of biological target 5 with effector template 7 which can be, but is not limited to, an antibody capable of initiating complement-mediated cytotoxicity, and promotion of the association of biological target 5 with effector template 7 which can be, but is not limited to, a cell that is capable of neutralizing biological target 5. In some embodiments, first functionality 4 and second functionality 6 of heterobifunctional ligand 2 recognize and bind biological target 5 and effector 7, which are two different membrane-bound protein receptors that exist as multiple copies on the same cell. In other embodiments, biological target 5 and effector template 7 are receptors on separate cells.

The term “detecting” refers to the use of multivalent heterobifunctional polymer 1 to determine whether a certain biological target is present in an organism or in an environment. This can be particularly advantageous in the area of diagnostics, which will be discussed further below.

In many cases, the identity of both first functionality 4 and second functionality 6 is known from the available literature. If their identities are not known for an intended application, effective first and second functionalities can be identified by screening libraries of known compounds or they can be rationally designed based on any available structural data for the receptors.

First functionality 4 and second functionality 6 can be selected from the group that can include, but is not limited to, an amino acid, a peptide, a derivatized peptide, a monosaccharide, an oligosaccharide containing between 0 to about 20 monosaccharides, a nucleotide, a nucleotide analog, a polynucleotide, a polynucleotide analog, a cell nutrient, a vitamin, an antigenic determinant, a small drug-like compound, a hapten, an antibody or antibody fragment, a cell surface receptor, and combinations and analogs thereof.

Biological target 5 may be selected from the group that can include, but is not limited to, a multivalent receptor, a multivalent protein, a protein, a peptide, a derivatized peptide, an antibody, a membrane-bound receptor, a bacteria, a Gram-positive bacteria, a Gram-negative bacteria, unicellular parasites, archaebacteria, fungi, a viral particle, a bacterial toxin, viral lectins, a cancer cell, B cells, and combinations and analogs thereof.

Effector template 7 may be selected from the group that can include, but is not limited to, a multivalent receptor, a multivalent protein, a protein, a peptide, a derivatized peptide, an antibody, a membrane-bound receptor, and combinations and analogs thereof.

In one embodiment, when effector template 7 is an antibody, second functionality 6 can be any arbitrary hapten chosen using properties which can include, but are not limited to, high immunogenicity, low molecular weight and/or low toxicity.

In one embodiment, multivalent polymer 1, which includes one or more copies of heterobifunctional ligand 2, is provided wherein first functionality 4 binds a multivalent biological target. Multivalent biological targets include those that present multiple binding sites and can simultaneously bind to more than one ligand. The term “multivalent biological targets” is also meant to encompass structural motifs on a cell surface, such as the clustering of similar cell surface receptors.

In one embodiment, the effector template is serum amyloid P component. In another embodiment the effector template is a molecular or cellular component of the innate or adaptive immune system.

In one embodiment, first functionality 4 binds biological target 5 that is a bacterial toxin. Examples of bacterial toxins include, but are not limited to, Shiga or Shiga-like toxins, heat-labile enterotoxin, subtilase cytotoxin and cholera toxin. In some aspects, the Shiga toxin is expressed by an enterohemorrhagic E. coli such as O157:H7 E. coli serotype. In some aspects, first functionality 4 is a trisaccharide. In other embodiments, first functionality 4 binds a biological target that is a Gram positive bacteria.

In one embodiment, first functionality 4 binds a biological target that is a viral particle such as the influenza virus. In some aspects, first functionality 4 binds viral hemagglutinin neuraminidase (HN). In one embodiment, first functionality 4 is a neuraminic acid derivative. In another embodiments, first functionality 4 binds viral lectins.

Other biological targets and corresponding first functionalities include, but are not limited to, fimbriated E. coli having FimH adhesin surface groups which can interact with mannose groups; S. pneumoniae having pneumococcal surface adhesin A (PsaA) surface groups capable of binding to glucosamine N-acetyl (GlcNac) groups; choline binding protein A (CpbA) groups capable of binding to neuraminic acid (NeuAc) and lacto-N-neotetraose groups; α-enolase groups capable of binding to plasmin(ogen) groups; and P. aeruginosa having pilus adhesin surface groups capable of binding to GlcNAc, NeuAc, and lactose.

In one embodiment, first functionality 4 binds biological target 5 that is a cell surface receptor of a cancer cell. In some aspects, the cell surface receptor is also present in normal cells but is upregulated in cancer cells. In some aspects, biological target 5 is a folate receptor of a cancer cell.

In one embodiment, first functionality 4 binds biological target 5 that is an integrin. In one embodiment, the integrin is integrin αvβ3.

In one embodiment, first functionality 4 binds biological target 5 that is a sialoglycoprotein associated with a B cell lymphoma.

In one embodiment, first functionality 4 is a phospholipid such as cardiolipin capable of binding to B cells displaying immunoglobulin G (IgG) associated with antiphospholipid antibody syndrome.

In one embodiment, first functionality 4 is a 2,6-linked sialic acid-containing oligosaccharide.

In one embodiment, first functionality 4 is chlorotoxin, a peptide derived from the venom of the giant Israeli scorpion, capable of binding specifically to a tumor surface marker found in a vast majority of gliomas (Deshane, J. et al. 2003, J. Biol. Chem. 278 (6):4135-4144).

In one embodiment, first functionality 4 is a peptide containing Arginine-Glycine-Aspartic Acid (RGD) or a functional derivative or synthetic mimetic thereof. In some aspects, the RGD is a cyclopeptide.

In one embodiment, first functionality 4 binds biological target 5 that is an antibody involved in an autoimmune disease. In some aspects, the antibody mediates Guillain-Barré syndrome.

In one embodiment, second functionality 6 binds to effector template 7 that is serum amyloid P component (SAP).

In another embodiment, effector template 7 is a molecular or cellular component of the innate or adaptive immune system.

In one embodiment, effector template 7 is a T-cell, B-cell, or a natural killer cell. In one embodiment, second functionality 6 is a hapten that binds effector template 7 that is an antibody. In some aspects, the antibody is one that is raised in a patient previously immunized against a compound containing second functionality 6. In some aspects, second functionality 6 is a sulfonamide. In another embodiment, second functionality 6 is a sulfathiazole.

### Linkers

As discussed above, optional linker 3 covalently connects heterobifunctional ligand 2 to multivalent polymer 1. Optional linkers 9A, 9B and 9C may also be present. The length and chemical composition of linkers 3, 9A, 9B and 9C will vary according to the intended application and the nature of biological target 5 and effector template 7. The chemical composition of linkers 3, 9A, 9B and 9C may also vary widely depending on the environment surrounding the binding site of biological target 5 and/or effector template 7. Depending on the intended use, it may be advantageous to synthesize a hydrophobic, hydrophilic or amphipathic linker.

Linkers 3, 9A, 9B and 9C may be comprised of a wide variety of different groups, whose identity is dependent on the intended application. These groups include, but are not limited to, alkylene chains having from a plurality of methylene groups, wherein independently each methylene group is optionally replaced with a divalent moiety. Suitable divalent moieties include —O—, —S(O)ₙ—, —NR—, —C(O)NR—, —C(O)O—, —CRR′—, carbamate, urea, and thiourea moieties where n is 0, 1, or 2 and R is H or alkyl and R′ is H, alkyl, or alkyl substituted with a non-hydrogen substituent. Other groups include those containing ethylene glycol units. Still other groups include optionally replacing one or more methylene groups with a 1,4-phenylene moiety.

Of course, one skill in the art will appreciate that many other groups not listed above could be used to synthesize linkers 3, 9A, 9B, and 9C in order for linkers 3, 9A, 9B and 9C to possess the properties required to assist in the formation of stable ternary complexes. These groups should be seen as being included within the scope of this invention.

### Polymeric Backbone

The polymeric backbone of multivalent heterobifunctional polymer 1 can take many different forms depending on the intended application. Polymers that can be used in the present invention include those having acyclic, cyclic and/or arylene structures in the backbone wherein the heterobifunctional ligands are attached to or into the polymer backbone. Other suitable polymers include polyacrylamide, polymeric carbohydrates such as hydroxypropylmethylcellulose and carboxymethylcellulose, and acrylic acid based polymers such as polycarbophil, carbomer (acrylic acid polymer), poly(methylmethacrylate) acrylic acid/butyl acrylate copolymers, poly[N-(2-hydroxypropyl)methacrylamide] (HPMA), poly(amino acids), poly(aspartic acid), poly(glutamic acid), and poly(malic acid). Naturally occurring polymers which include, but are not limited to, dextrans, dextrins, agarose, amylose, hyaluronic acid, glycosaminoglycan and chitosans, may also be used. Of course, one of skill in the art will appreciate that any pharmaceutically acceptable polymer that can be covalently joined to a plurality of heterobifunctional ligands could be used to synthesize multivalent polymers of the present invention. These polymers should be seen as being included within the scope of this invention.

Polymers can be prepared by polymerizing monomers having a polymerizable group such as a terminal double bond attached to the heterobifunctional ligand. Co-polymers of the heterobifunctional monomers with non-functionalized monomers and/or non-functionalized monomers which alter and or improve the physical or biological properties such as the solubility or stability of the polymer can also be made and employed. Methods for preparing polymers also include, but are not limited to, ring-opening metathesis polymerization (ROMP). The degree of loading of the polymer with heterobifunctional ligands depends on the ratio of functionalized and unfunctionalized monomers and on the nature and reactivities of the monomers. In some aspects, the polymers have 10-20 repeating units per heterobifunctional ligand. In other aspects the polymers have 20 or more repeating units per heterobifunctional ligand.

The terms “connection points” or “connected” when applied to attaching a heterobifunctional ligand to a polymer should be construed broadly. As discussed above, heterobifunctional ligands may be connected to the polymer through optional linkers or may be connected directly to the polymer. The type of connection will depend on the nature of the heterobifunctional ligands, the polymer and the intended use. In one embodiment, the connections are covalent.

### Administration and Pharmaceutical Composition

In general, the polymers of this invention will be administered in a therapeutically effective amount by any of the accepted modes of administration for agents that serve similar utilities. The actual amount of the polymer will depend upon numerous factors such as the polymer pharmacokinetics, the severity of the disease to be treated, the age and relative health of the subject, the potency of the polymer used, the route and form of administration, and other factors. The drug can be administered more than once a day, preferably once or twice a day. All of these factors are within the skill of the attending clinician.

In general, polymers of this invention will be administered as pharmaceutical compositions by any one of the following routes: systemic (e.g., transdermal, intranasal or by suppository), parenteral (e.g., intramuscular, intravenous or subcutaneous), intrathecal, or oral administration. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration and bioavailability of the drug substance. For delivery via inhalation, the polymer can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI).

The compositions are comprised of, in general, a polymer of the invention in combination with at least one pharmaceutically acceptable excipient. Acceptable excipients are non-toxic, aid administration, and do not adversely affect the therapeutic benefit of the polymer. Such excipient may be any solid, liquid, semi-solid or, in the case of an aerosol composition, a gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a polymer of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

### Therapeutic and Non-Therapeutic Applications

As discussed above, the identity of first functionality 4 and second functionality 6 can vary widely depending on the identity of biological target 5 and effector template 7. Multivalent heterobifunctional polymers of the present invention may be used for both therapeutic and non-therapeutic applications.

There are many different possible therapeutic applications for multivalent heterobifunctional polymer 1 of the present invention. Listed below are only a few of the possible therapeutic applications, which are not meant to be limiting.

In one embodiment, multivalent heterobifunctional polymer 1 can be used to remove bacterial toxins, which can include, but are not limited to, Shiga or Shiga-like toxins, heat-labile enterotoxin, subtilase cytotoxin, and cholera toxin.

In one embodiment, multivalent heterobifunctional polymer 1 can be used to remove viral particles, which can include, but is not limited to, the influenza virus.

In one embodiment, multivalent heterobifunctional polymer 1 can be used to remove fimbriated E. coli, S. pneumoniae, choline binding protein A groups, α-enolase groups, and P. aeruginosa.

In one embodiment, multivalent heterobifunctional polymer 1 can be used to target cancer cells.

In one embodiment, multivalent heterobifunctional polymer 1 can be used to target an integrin. In one embodiment, the integrin is integrin αvβ3.

In one embodiment, multivalent heterobifunctional polymer 1 can be used to target a sialoglycoprotein associated with a B cell lymphoma.

In one embodiment, multivalent heterobifunctional polymer 1 can be used to target a phospholipid, which can include, but is not limited to, cardiolipin that can bind to B cells displaying IgG associated with antiphospholipid antibody syndrome.

In one embodiment, multivalent heterobifunctional polymer 1 can be used to target gliomas.

In one embodiment, multivalent heterobifunctional polymer 1 can be used to eliminate antibodies involved in autoimmune diseases, which can include, but is not limited to, Guillain-Barré syndrome.

In one embodiment, a method of targeted immunotherapy is provided comprising administering an effective amount of a polymer of the invention wherein second functionality 6 is a hapten, such that administration of said polymer initiates immune recognition by pre-existing antibodies. In some aspects, the antibodies are raised in a patient prior to commencing a treatment by administering a compound displaying second functionality 6.

In one embodiment, multivalent heterobifunctional polymer 1 can be used in the treatment of hereditary and acquired metabolic disorders.

Multivalent heterobifunctional polymers of the present invention can also be used for various non-therapeutic applications. The use of these polymers can be advantageous in the area of diagnostics. In one embodiment, multivalent heterobifunctional polymer 1 may be used to detect the presence of Stx1 in ground water. In another embodiment, multivalent heterobifunctional polymer 1 may be used to detect the presence of antibodies in blood. This may be advantageous in the diagnosis of many diseases, which can include, but is not limited to, cancer. In one embodiment, multivalent heterobifunctional polymer 1 can be used in tumour imaging. Of course, as will be appreciated by one of skill in the art, these non-therapeutic applications can be made possible by selecting and pre-arranging first functionality 4 and second functionality 6 on heterobifunctional ligands 2 so that the formation of specific ternary complexes is optimized.

### Materials, Methods and Examples

The following MATERIALS AND METHODS were used in the examples that follow. These materials and methods are for illustrative purposes only and are not to be construed as limiting the scope of the invention in any way. One of skill in the art will appreciate that several modifications and substitutions can be made without affecting the scope of the invention. More specifically, these include modifications and substitutions in the specific techniques and reaction conditions listed below.

### General Methods

Optical rotations were measured on a Perkin-Elmer 241 polarimeter in a 10 cm cell at ambient temperature. Analytical TLC was performed on silica gel 60-F254 (Merck) with detection by quenching of fluorescence and/or by charring with 10% H₂SO₄ in ethanol solution followed by heating at 180° C. Column chromatography was performed on silica gel 60 (Merck, 40-60 μm), and solvents were used as supplied. ¹H-NMR spectra were recorded at 400, 500 or 600 MHz (Varian) in CDCl₃ (referenced to residual CHCl₃ at δ_{H} 7.24 ppm) or in D₂O (referenced to external acetone at δ_{H} 2.225 ppm). J values are given in Hz. All commercial reagents were used as supplied.

In order that the invention be more fully understood, the following examples are set forth. These examples are for illustrative purposes only and are not to be construed as limiting the scope of the invention in any way. Moreover, these examples are not intended to exclude equivalents and variations of the present invention, which are apparent to one skilled in the art.

### Example 1

### Synthesis of compound 3,6,9,12,15,18-hexa-oxa-henicos-20-enyl 4-O-[4-O-(<-D-galactopyranosyl)-β-D-galactopyranosyl]-β-D-glucopyranoside (1)

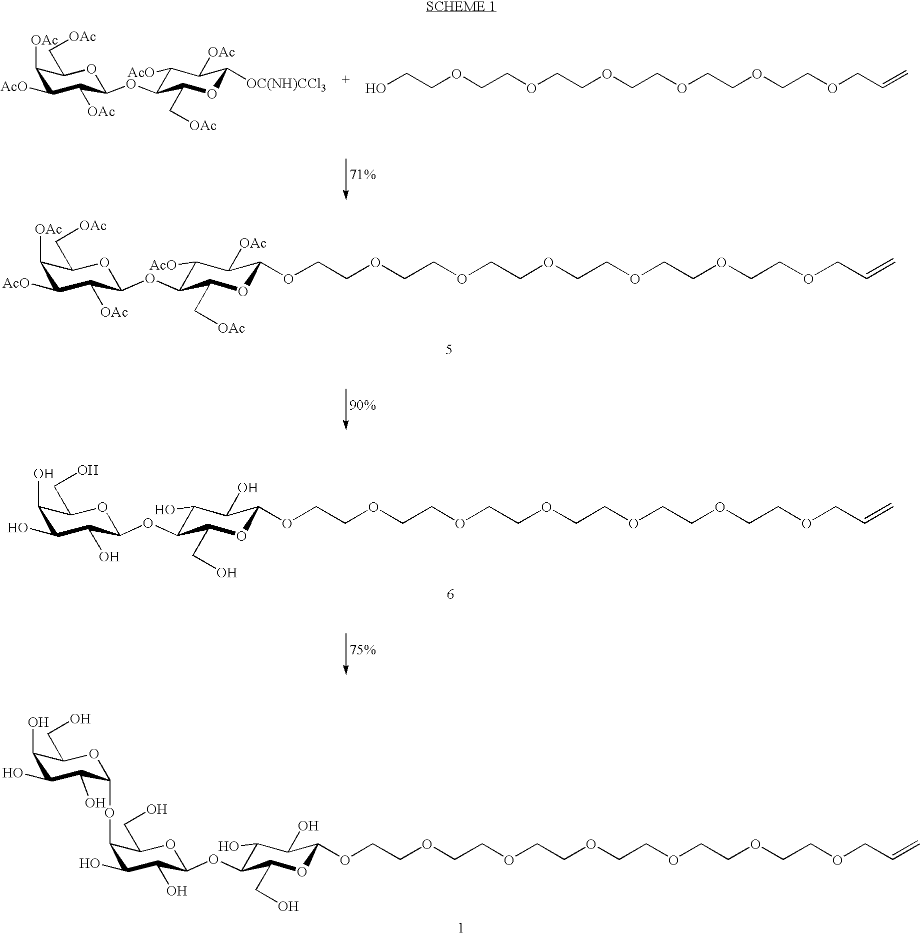

The known lactose imidate donor was coupled with monoallylated hexa(ethylene glycol) in the presence of boron trifluoride etherate to provide lactoside 5 in 71% yield. The deacetylation of 5 under Zemplen conditions gave heptaol 6 in 90% yield. The 6 was then galactosylated enzymatically using α-(1,4)-galactosyltransferase/UDP-4′-Gal-epimerase to provide target compound 1 in 75% yield.

3,6,9,12,15,18-hexa-oxa-henicos-20-enyl 2,3,6-tri-O-acetyl-4-O-(2,3,4,6-tetra-Oacetyl-β-D-galactopyranosyl)-β-D-glucopyranoside (5). The lactose hexaacetate trichloroacetimidate donor (1.8 g, 2.3 mmol, α:β as 9:1 mixture), monoallyl hexa(ethylene glycol) (0.64 g, 2.0 mmol) and activated 4 Å molecular sieves (1.5 g) were stirred for one hour in dry dichloromethane (20 mL). Then the mixture was cooled down to 0° C. and BF₃Et₂O (0.3 mL) was added dropwise. After TLC indicated the reaction to be completed, it was neutralized with Et₃N, filtered through Celite and concentrated. Chromatography of the residue on the silica gel provided the title compound 5 (1.3 g, 71% yield). ¹H-NMR (CDCl₃): δ_{H} 5.89 (m, 1H, allyl), 5.32 (dd, 1H, J 1.0 Hz, 3.5 Hz, H-4′), 5.27 (m, 1H, allyl), 5.23 (m, 1H, allyl), 5.15 (m, 2H, H-3 and allyl), 5.08 (dd, 1H, J 8.0 Hz, 10.5 Hz, H-2′), 4.93 (dd, 1H, J 3.5 Hz, 10.5 Hz, H-3′), 4.86 (dd, 1H, J 8.0 Hz, 9.5 Hz, H-2), 4.53 (d, 1H, J 7.5 Hz, H-1), 4.46 (m, 2H, H-1′ and H-6a), 4.07 (m, 2H, H-6b and H-6a′), 4.00 (m, 2H, allyl), 3.84 (m, 2H, H-5′), 3.77 (t, 1H, J 9.5 Hz, H-4), 3.69 (m, 1H, H-6b′), 3.57-3.65 (m, 15H), 2.13 (s, 3H, OAc), 2.10 (s, 3H, OAc), 2.04 (s, 3H, OAc), 2.02 (s, 3H, OAc), 2.02 (s, 3H, OAc), 2.01 (s, 3H, OAc), 1.94 (s, 3H, OAc). ESI-HRMS m/z 963.36814 ([M+Na]⁺, C₄₁H₆₄O₂₄Na⁺ requires 963.36798).

3,6,9,12,15,18-hexa-oxa-henicos-20-enyl 4-O-(β-D-galactopyranosyl)-β-D-glucopyranoside (6). The lactoside 5 (1.3 g, 1.4 mmol) was dissolved in dry MeOH (20 mL) and MeONa (3 mL, 0.5 M solution) was added. The mixture was stirred overnight and then neutralized with Amberlite H⁺, filtered and concentrated to provide title compound 6 (0.8 g, 90% yield). ¹H-NMR (D₂O): δ_{H} 5.95 (m, 1H, allyl), 5.33 (m, 1H, allyl), 5.26 (m, 1H, allyl), 4.50 (d, 1H, J 7.8 Hz, H-1′), 4.44 (d, 1H, J 7.8 Hz, H-1), 4.06 (m, 3H, allyl), 3.97 (m, 1H), 3.92 (m, 1H), 3.58-3.84 (m, 31H), 3.53 (m, 1H, H-2), 3.33 (m, 1H, H-2′). ESI-HRMS m/z 669.29389 ([M+Na]⁺, C₂₇H₅₀O₁₇Na⁺ requires 669.29402).

3,6,9,12,15,18-hexa-oxa-henicos-20-enyl 4-O-[4-O-(α-D-galactopyranosyl)-β-D-galactopyranosyl]-β-D-glucopyranoside (1). The lactoside 6 (0.11 g, 0.17 mmol) was dissolved in 4 mL of H₂O, HEPES buffer [1.25 mL, 1.6 M, 10 mM MnCl₂, bovine serum albumine (BSA, 0.8 mg/mL), pH 8], DTT solution (100 mM, 0.32 mL) and alkaline phosphatase (63 μl). To the mixture UDP-Glc (0.13 g) was added, followed by α-(1,4)-galactosyltransferase/UDP-4′-Gal-epimerase (0.625 mL). The reaction was incubated at 37° C. overnight and then chromatographed on C₁₈ to afford title compound 1 (0.1 g, 75% yield). ¹H-NMR (D₂O): δ_{H} 5.95 (m, 1H, allyl), 5.34 (m, 1H, allyl), 5.27 (m, 1H, allyl), 4.94 (d, 1H, J 3.6 Hz, H-1″), 4.51 (d, 1H, J 8.4 Hz, H-1′), 4.51 (d, 1H, J 7.8 Hz, H-1), 4.35 (m, 1H), 3.56-4.07 (m, 42H), 3.33 (t, 1H, J 8.4 Hz, H-2′). ESI-HRMS m/z 831.34649 ([M+Na]⁺, C₃₃H₆₀O₂₂Na⁺ requires 831.34685).

### Example 2

### Synthesis of 25-[(cis)-2-hydroxycarbonyl-2-methyl-[1,3]dioxane-5-yloxy]-24-(R,S)-hydroxy-4,7,10,13,16,19,22-hepta-oxa-pentadecos-1-ene (2)

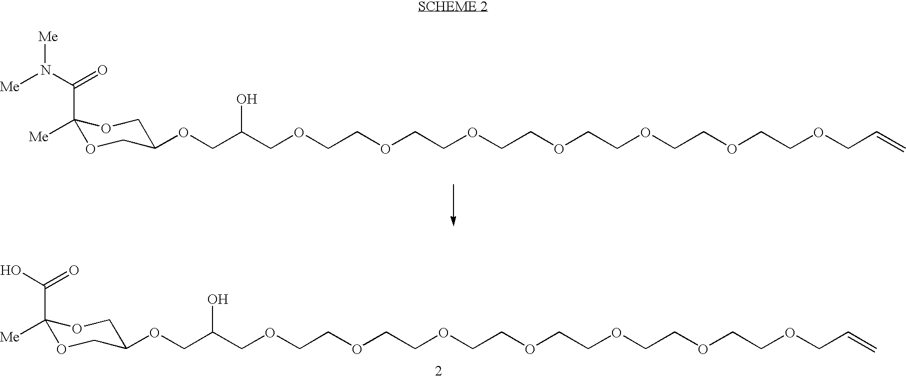

To a solution of 25-[(cis)-2-Dimethylaminocarbonyl-2-methyl-[1,3]dioxane-5-yloxy]-24-(R,S)-hydroxy-4,7,10,13,16,19,22-hepta-oxa-pentadecos-1-ene (125 mg, 0.214 mmol) in methanol (4.5 mL), 4 M aqueous NaOH (268 μL) was added. The reaction mixture was stirred at 80° C. overnight. On the following day, additional 4 M NaOH (134 μL) was added and the mixture was left at 80° C. overnight. On the following day, NMR and TLC confirmed that the hydrolysis of the amide was complete. The mixture was diluted with methanol and deionized with Dowex H⁺ resin, filtered and concentrated. The dry residue was dissolved in water and freeze-dried to provide the product as a syrup (97 mg; 84%). ¹H-NMR (D₂O) δ: 5.99-5.92 (m, 1 H, HC═CH₂), 5.36-5.26 (m, 2H, HC═CH₂), 4.18 (dd, 2H, J 4.7 Hz, J 11.4 Hz, H-4e, H-6e), 4.07 (d, 2H, J 5.9 Hz, CH₂—CH═CH₂), 3.94-3.92 (m, 1H, CH), 3.74-3.65 (m, 26H, H-5, OCH₂), 3.70-3.60 (m, 5H, H-4a, H-6a, OCH₂), 1.50 (s, 3H, CH₃). Electrospray ionization HRMS, calcd for C₂₄H₄₄O₁₃Na (M+Na): m/z 563.26741, found: m/z 563.26776.

### Example 3

### Synthesis of 24-(R,S)-[4-O-(4-O-(α-D-Galactopyranosyl)-β-D-galactopyranosyl)-β-D-glucopyranosyloxy]-25-[(cis)-2-carboxyl-2-methyl-[1,3]dioxane-5-yloxy]-4,7,10,13,16,19,22-hepta-oxa-pentacos-1-ene (3)

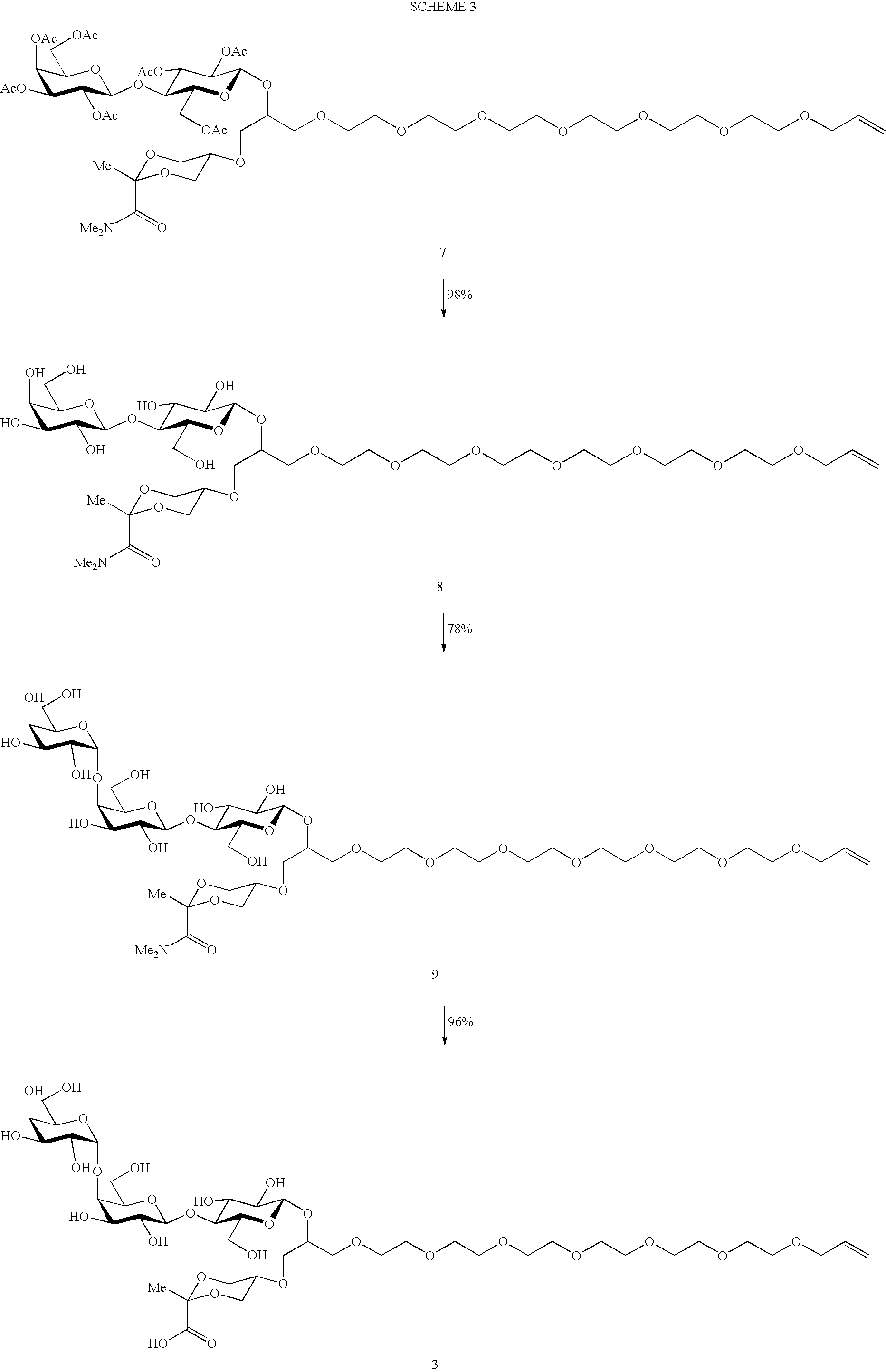

The known lactoside 7 (Solomon et al. (Organic Letters 2005, 7, 4369-4372)) was deacetylated under Zemplen conditions to provide glycoside 8 in 98% yield. The 8 was galactosylated enzymatically using α-(1,4)-galactosyltransferase/UDP-4′-Gal-epimerase to provide trisaccharide 9 in 78% yield. The hydrolysis of amide group of 9 under basic conditions afforded target compound 10 in 96% yield.

24-(R,S)-[4-O-(β-D-galactopyranosyl)-β-D-glucopyranosyloxy]-25-[(cis)-2-dimethylaminocarbonyl-2-methyl-[1,3]dioxane-5-yloxy]-4,7,10,13,16,19,22-hepta-oxa-pentacos-1-ene (8). The compound 7 (0.33 g, 0.3 mmol) (Solomon et al (Organic Letters 2005, 7, 4369-4372)) was dissolved in dry MeOH (10 mL) and MeONa (1 mL of 0.5 M solution) was added. After stirring overnight at room temperature the mixture was neutralized with Amberlite (H+) resin, filtered, concentrated and dried in vacuum to afford title compound 8 (0.24 g, 98% yield). ¹H-NMR (D₂O): δ_{H} 5.94 (m, 1H, allyl), 5.34 (m, 1H, allyl), 5.27 (m, 1H, allyl), 4.60 (m, 1H, H-1′), 4.44 (m, 1H, H-1), 4.20 (m, 2H), 4.07 (m, 3H, allyl), 3.96 (m, 1H), 3.92 (m, 1H, H-4′), 3.61-3.82 (m, 39H, OMe), 3.53 (m, 4H), 3.31 (m, 1H, H-2′), 3.26 (s, 3H, NMe), 3.00 (s, 3H, NMe), 1.52 (m, 3H, C-Me). ESI-HRMS m/z 914.42097 ([M+Na]⁺, C₃₈H₆₉NO₂₂Na⁺ requires 914.42035).

24-(R,S)-[4-O-(4-O-(α-D-Galactopyranosyl)-β-D-galactopyranosyl)-β-D-glucopyranosyloxy]-25-[(cis)-2-dimethylaminocarbonyl-2-methyl-[1,3]dioxane-5-yloxy]-4,7,10,13,16,19,22-hepta-oxa-pentacos-1-ene (9). The lactoside 8 (0.24 g, 0.27 mmol) was dissolved in 8 mL of H₂O, HEPES buffer [2.5 mL, 1.6 M, 10 mM MnCl₂, bovine serum albumin (BSA, 0.8 mg/mL), pH 8], DTT solution (100 mM, 0.63 mL) and alkaline phosphatase (125 μL). To the mixture UDP-Glc (0.25 g) was added, followed by α-(1,4)-galactosyltransferase/UDP-4′-Gal-epimerase (1.25 mL). The reaction was incubated at 37° C. overnight and then chromatographed on C₁₈ to afford title compound 9 (0.22 g, 78% yield). ¹H-NMR (D₂O): δ_{H} 5.94 (m, 1H, allyl), 5.34 (m, 1H, allyl), 5.27 (m, 1H, allyl), 4.94 (d, 1H, J 3.6 Hz, H-1″), 4.60 (m, 1H, H-1′), 4.50 (m, 1H, H-1), 4.34 (t, 1H, J 6.0 Hz, H-5″), 4.20 (m, 2H), 3.49-4.08 (m, 57H), 3.31 (m, 1H, H-2′), 3.26 (s, 3H, NMe), 3.00 (s, 3H, NMe), 1.52 (m, 3H, C-Me). ESIHRMS m/z 1076.47343 ([M+Na]⁺, C₄₄H₇₉NO₂₇Na⁺ requires 1076.47317).

24-(R,S)-[4-O-(4-O-(α-D-Galactopyranosyl)-β-D-galactopyranosyl)-β-D-glucopyranosyloxy]-25-[(cis)-2-carboxyl-2-methyl-[1,3]dioxane-5-yloxy]-4,7,10,13,16,19,22-hepta-oxa-pentacos-1-ene (3). A solution of 9 (0.15 g, 0.14 mmol) and NaOH (5 equivalents) was stirred 3 days at 80° C. and the progress was followed by NMR, the mixture was neutralized with Amberlite H⁺ resin, filtered and concentrated to give title compound 3 (0.14 g, 96% yield). ¹HNMR (D₂O): δ_{H} 5.95 (m, 1H, allyl), 5.34 (m, 1H, allyl), 5.27 (m, 1H, allyl), 4.94 (m, 1H, H-1″), 4.60 (d, 1H, J 7.8 Hz, H-1′), 4.50 (m, 1H, H-1), 4.34 (t, 1H, J 6.0 Hz, H-5″), 4.20 (m, 2H), 3.55-4.09 (m, 57H), 3.31 (m, 1H, H-2′), 1.50 (m, 3H, C-Me). ESI-HRMS m/z 1049.42593 ([M+Na]⁺, C₄₂H₇₄O₂₈Na⁺ requires 1049.42588).

### Example 4

### Synthesis of 4-O-[4-O-(α-D-Galactopyranosyl)-β-D-galactopyranosyl]-6-N-(4-pentenylcarbamoyl)-1,2-O—[(S)-1-(carboxy)ethylidene]-α-D-glucopyranose (4)

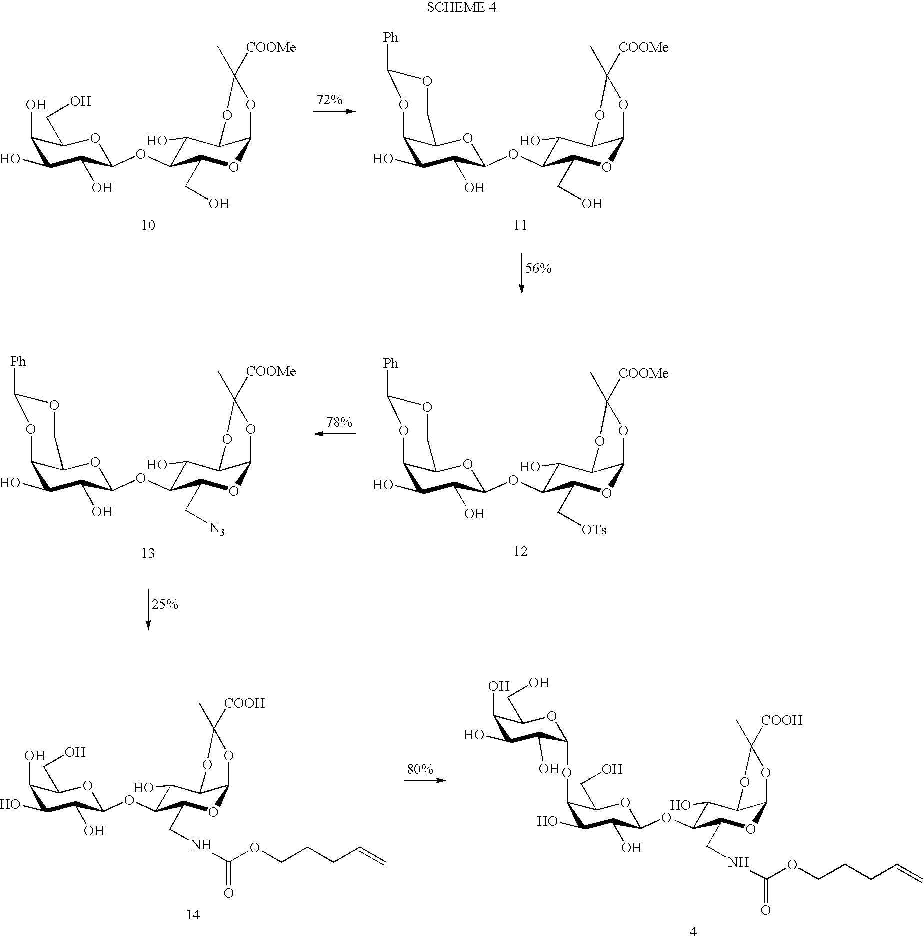

The known compound 10 was treated with benzaldehyde dimethyl acetal in presence of catalytic CSA to provide benzylidene 11 in 72% yield. The remaining primary hydroxyl of 11 was selectively tosylated using tosyl chloride in pyridine to afford triol 12 in 56% yield. The subsequent substitution of tosyl group by azide provided compound 13 in 78% yield. Hydrogenation of 13 followed by subsequent coupling of resulting amine with p-nitrophenyl 4-pentenyl carbonate and removal of benzylidene protecting group with 80% acetic acid afforded carbamate 14. The disaccharide 14 was galactosylated enzymatically using α-(1,4)-galactosyltransferase/UDP-4′-Gal-epimerase to provide desired trisaccharide 4.

4-O-(4,6-O-Benzylidene-β-D-galactopyranosyl)-1,2-O—[(S)-1-(methoxycarbonyl)ethylidene]-α-D-glucopyranose (11). The hexaol 10 (2.19 g, 4.25 mmol) was dissolved in dry acetonitrile (24 mL) and benzaldehyde dimethyl acetal (1.95 mL, 3 equivalents) and camphorsulfonic acid (catalytic amount) were subsequently added. After TLC indicated the reaction completed, the acid was neutralized with triethylamine and the solvent was evaporated. Chromatography of the residue on silica gel (DCM:MeOH=20:1) afforded title compound 11 (1.59 g, 72%). ¹H-NMR (CDCl₃): δ_{H} 7.35-7.49 (m, 5H, aromatic protons), 5.79 (d, 1H, J_{1,2} 5.4 Hz, H-1), 5.47 (s, 1H, C₆H₅CH), 4.45 (d, 1H, J 7.8 Hz, H-1′), 4.23 (m, 1H, H-6a′), 4.15 (t, 1H, J_{2,3} 4.8 Hz, H-2), 4.07 (d, 1H, J_{3′,4′} 3.6 Hz, H-4′), 3.93-4.00 (m, 3H, H-3, H-6a, H-6b′), 3.73-3.82 (m, 3H, H-5, H-6b, H-2′), 3.75 (s, 3H, COOMe), 3.69 (t, 1H, J 8.4 Hz, H-4), 3.62 (dd, 1H, J_{2′,3′} 9.6 Hz, J_{3′,4′} 3.6 Hz, H-3′), 3.47 (bs, 1H, H5′), 1.68 (s, 3H, CH₃). ESI-HRMS m/z 537.15750 ([M+Na]⁺, C₂₃H₃₀O₁₃Na⁺ requires 537.15786).

4-O-(4,6-O-Benzylidene-β-D-galactopyranosyl)-6-O-tosyl-1,2-O—[(S)-1-(methoxycarbonyl)ethylidene]-α-D-glucopyranose (12). The compound 11 (1. g, 1.9 mmol) was dissolved in dry pyridine (8 mL). After the mixture was cooled down to 0° C., tosyl chloride (0.3 g) followed by 4-dimethylaminopyridine (25 mg) were added. After 3 h more tosyl chloride (0.25 g) was added. After 16 h in fridge (+5° C.) the reaction was quenched with methanol. After solvents were evaporated residue was chromatographed on silica gel (DCM:MeOH=20:1) to provide title compound 12 (0.733 g, 56%). ¹H-NMR (CDCl₃): δ_{H} 7.32-7.80 (m, 9H, aromatic protons), 5.69 (d, 1H, J_{1,2} 5.5 Hz, H-1), 5.53 (s, 1H, C₆H₅CH), 4.50 (dd, 1H, J 3.5 Hz, 11.0 Hz, H-6a), 4.46 (d, 1H, J_{1′,2′} 8.0 Hz, H-1′), 4.26 (m, 2H, H-6b, H-6a′), 4.19 (d, 1H, J_{3′,4′} 3.5 Hz, H-4′), 4.15 (t, 1H, J 4.5 Hz, H-2), 4.05 (dd, 1H, J 2.0 Hz, 12.5 Hz, H-6b′), 3.97 (m, 2H, H-3 and H-5), 3.74 (m, 4H, H-2′ and COOMe), 3.66 (m, 2H, H-4 and H-3′), 3.56 (bs, 1H, H-5′), 2.44 (s, 3H, MePh), 1.65 (s, 3H, CH₃). ESI-HRMS m/z 691.16689 ([M+Na]⁺, C₃₀H₃₆O₁₅SNa⁺ requires 691.16671).

6-Azido-4-O-(4,6-O-benzylidene-β-D-galactopyranosyl)-1,2-O—[(S)-1-(methoxycarbonyl)ethylidene]-α-D-glucopyranose (13). The triol 12 (0.721 g, 1.078 mmol) was dissolved in dry DMF (7 mL) and sodium azide (0.218 g, 3.35 mmol) was added. The reaction mixture was stirred at 60° C. for 18 hours. The solvent was evaporated and the residue was taken up in DCM, filtered, coevaporated twice with toluene. Chromatography of residue on silica gel (hexane:acetone=1:1) provided 13 (0.424 g, 78%). ¹H-NMR (CDCl₃): δ_{H} 7.37-7.49 (m, 5H, aromatic protons), 5.85 (d, 1H, J_{1,2} 5.0 Hz, H-1), 5.53 (s, 1H, C₆H₅CH), 4.41 (d, 1H, J_{1′,2′} 8.0 Hz, H-1′), 4.29 (dd, 1H, J 1.5 Hz, 13.0 Hz, H-6a′), 4.19 (m, 2H, H-2 and H-4′), 4.07 (dd, 1H, J 2.0 Hz, 13.0 Hz, H-6b′), 3.99 (m, 1H, H-5), 3.93 (dd, 1H, J 5.0 Hz, 8.0 Hz, H-3), 3.77 (s, 3H, COOMe), 3.73 (dd, 1H, J 7.8 Hz, 9.7 Hz, H-2′), 3.63-3.70 (m, 4H, H-3′, H-4, H-6a, H-6b), 3.57 (bd, 1H, H-5′), 1.71 (s, 3H, CH₃). ESI-HRMS m/z 562.16445 ([M+Na]⁺, C₂₃H₂₉N₃O₁₂Na⁺ requires 562.16435).

4-O-(β-D-Galactopyranosyl)-6-N-(4-pentenylcarbamoyl)-1,2-O—[(S)-1-(carboxy)ethylidene]-α-D-glucopyranose (14). The azide 13 (0.284 g, 0.526 mmol) was catalytically hydrogenated using Pd(OH)₂/C (200 mg) in MeOH (6 mL) for 24 hours. The catalyst was removed by filtration, the solvent was evaporated and the residue was dried. Then the resulting amine was dissolved in dry acetonitrile (6 mL) and p-nitrophenyl 4-pentenyl carbonate (0.16 g) was added. After reaction was completed, the solvent was evaporated and dissolved in 80% AcOH (10 mL) The mixture was stirred for 2 h at 80° C. then concentrated and chromatographed on silica gel (DCM:MeOH=10:1-6:1) to give 14 (70 mg, 25%). ¹H-NMR (D₂O): δ_{H} 5.91 (m, 1H, H_{d} of pentenyl), 5.67 (d, 1H, J_{1,2} 5.0 Hz, H-1), 5.02-5.11 (m, 2H, Hₑ of pentenyl), 4.50 (d, 1H, J_{1′,2′} 7.5 Hz, H-1′), 4.39 (m, 1H, H-3), 4.23 (m, 1H, H-2), 4.10 (m, 2H, Hₐ of pentenyl), 3.92 (d, 1H, J 3.0 Hz, H-4′), 3.88 (m, 1H, H-5), 3.74-3.83 (m, 2H, H-6a′ and H-6b′), 3.70 (dd, 1H, J 4.0 Hz, 8.0 Hz), 3.65 (m, 2H, H-4 and H-5′), 3.56 (m, 2H, H-6a and H-2′), 3.34 (m, 1H, H-6b), 2.15 (m, 2H, H_{c} of pentenyl), 1.74 (m, 2H, H_{b} of pentenyl), 1.66 (s, 3H, CH₃).

4-O-[4-O-(α-D-Galactopyranosyl)-β-D-galactopyranosyl]-6-N-(4-pentenylcarbamoyl)-1,2-O—[(S)-1-(carboxy)ethylidene]-α-D-glucopyranose (4). The carbamate 14 (60.8 mg, 0.116 mmol) was dissolved in 1.34 mL of H₂O, HEPES buffer [0.396 mL, 1.6 M, 10 mM MnCl₂, bovine serum albumin (BSA, 0.8 mg/mL), pH 8], DTT solution (100 mM, 0.1 mL) and alkaline phosphatase (10 μL). To this mixture UDP-Glc (0.112 g, 1.58 eq) was added, followed by α-(1,4)-galactosyltransferase/UDP-4′-Gal-epimerase (0.198 mL). The reaction was incubated at 37° C. for 21 h then ultra-centrifuged, treated with DOWEX (H⁺) and chromatographed on C-18 HPLC (water:MeOH:0.1% TFA, eluted with 25-30% MeOH) to afford the title compound 4 (63.3 mg, 80%). ¹H-NMR (D₂O): δ_{H} 5.88 (m, 1H, H_{d} of pentenyl), 5.71 (d, 1H, J_{1,2} 5.0 Hz, H-1), 5.00-5.09 (m, 2H, Hₑ of pentenyl), 4.94 (d, 1H, J 4.0 Hz, H-1″), 4.55 (d, 1H, J_{1′,2′} 8.0 Hz, H-1′), 4.37 (m, 2H, H-3 and H-4″), 4.30 (t, 1H, J 4.0 Hz, H-2), 4.08 (m, 2H, Hₐ of pentenyl), 4.02 (m, 2H, H-4′), 3.65-3.92 (m, 10H, H-2″, H-3″, H-3′, H-6a), 3.53-3.60 (m, 2H, H-5 and H-2′), 3.32 (dd, 1H, J 7.5 Hz, 10.0 Hz, H-6b), 2.12 (m, 2H, H_{c} of pentenyl), 1.71 (m, 2H, H_{b} of pentenyl), 1.70 (s, 3H, CH₃). ESI-HRMS m/z 708.23206 ([M+Na]⁺, C₂₇H₄₃NO₁₉Na⁺ requires 708.23215).

### Example 5

### Preparation of Prior Art Polymer PPM Having Two Independent Unifunctional Ligands

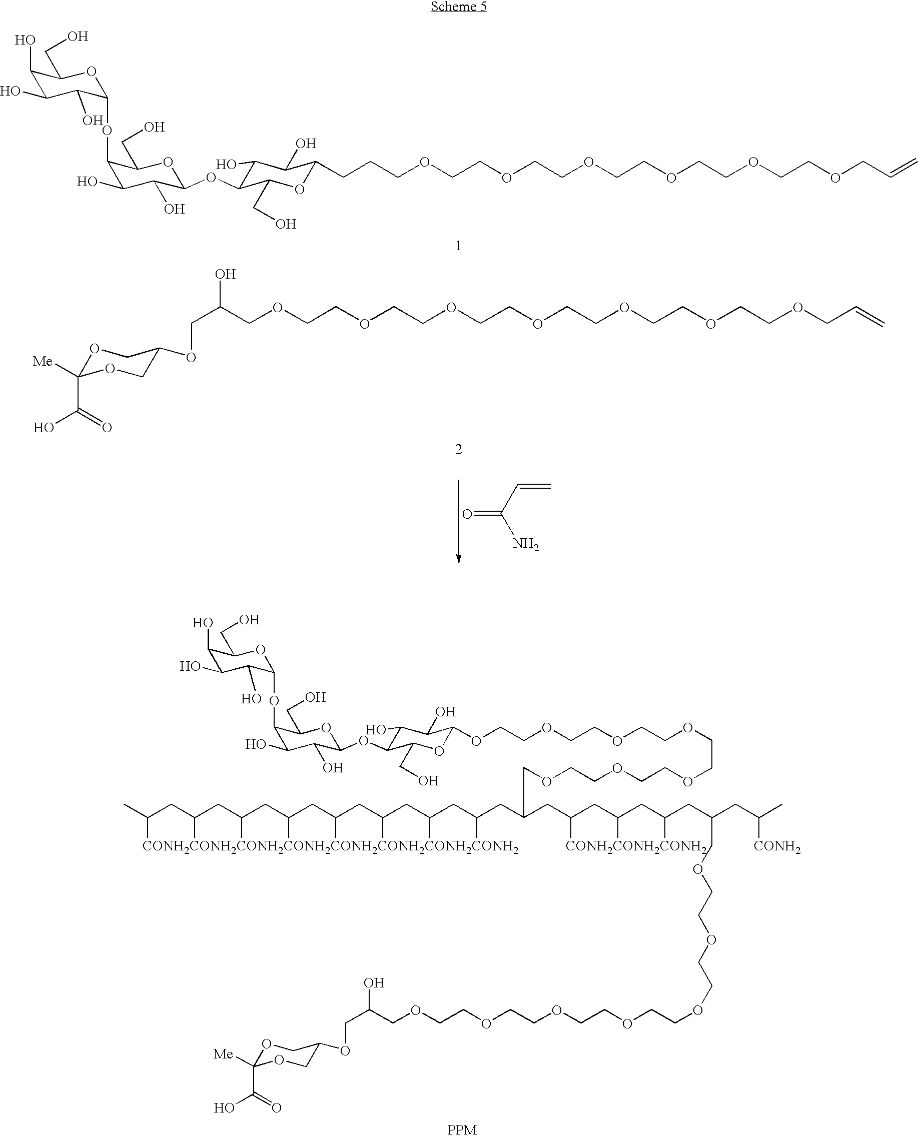

Scheme 5 shown above illustrates the incorporation of unifunctional ligands 1 and 2 via radical polymerization. Compound 1 (3,6,9,12,15,18-hexa-oxa-henicos-20-enyl 4-O-[4-O-(α-D-galactopyranosyl)-β-D-galactopyranosyl]-β-D-glucopyranoside) targets Shiga-like toxin and compound 2 targets SAP.

To a solution of acrylamide (17.1 mg, 0.25 mmol), monomer 1 (81 mg, 0.1 mmol) and monomer 2 (54 mg, 0.1 mmol) in degassed water (1 mL) a solution of sodium persulfate (1 mg) in of water (10 μL) was added. The solution was spurged with argon and TEMED (12 μL) was added. The mixture was incubated for 16 h then dialyzed and freezed dried to give 13 mg of PPM. NMR data (not shown) indicate ˜4.9% incorporation of each ligand.

### Example 6

### Preparation of Multivalent Polymer PPI, Having a Plurality of Heterobifunctional Ligands, where the Two Functionalities are Joined to a Common Atom Via Linkers

Scheme 6 shown below illustrates the incorporation of a heterobifunctional ligand via radical polymerization. Compound 3 (24-(R,S)-[4-O-(4-O-(α-D-galactopyranosyl)-β-D-galactopyranosyl)-β-D-glucopyranosyloxy]-25-[(cis)-2-carboxyl-2-methyl-[1,3]dioxane-5-yloxy]-4,7,10,13,16,19,22-hepta-oxa-pentacos-1-ene) targets Shiga toxin through the trisaccharide moiety and the moiety comprising the cyclic pyruvate of glycerol targets SAP.

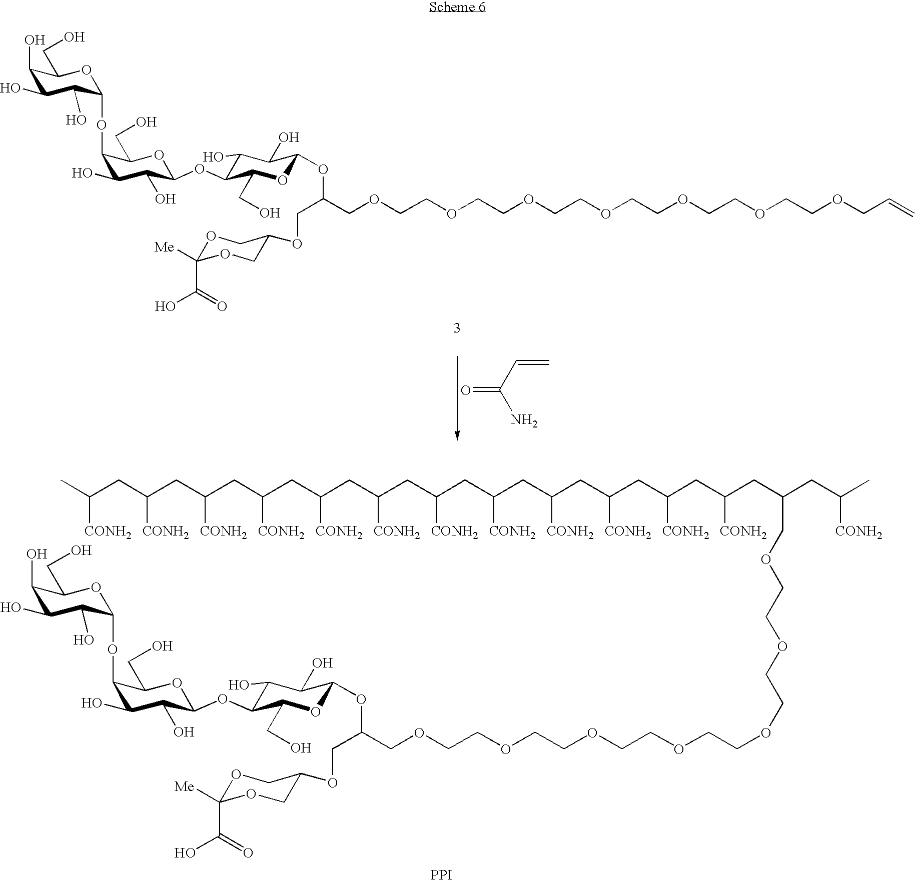

To a solution of acrylamide (34.2 mg, 0.5 mmol) and monomer 3 (103 mg, 0.1 mmol) in degassed water (1 mL) a solution of sodium persulfate (1 mg) in of water (10 μL) was added. The solution was spurged with argon and TEMED (12 μL) was added. The mixture was incubated for 16 h then dialyzed and freezed dried to give 24 mg of PPI. NMR data (not shown) indicate ˜2.6% of sugar monomer incorporation.

### Example 7

### Preparation of Multivalent Polymer EPI-156, Having a Plurality of Heterobifunctional Ligands, where the Two Functionalities are Joined to One Another without the Use of a Common Atom or Linker

Scheme 7 shown below illustrates the incorporation of a heterobifunctional ligand via radical polymerization. Compound 4 (4-O-[4-O-(α-D-Galactopyranosyl)-β-D-galactopyranosyl]-6-N-(4-pentenylcarbamoyl)-1,2-O—[(S)-1-(carboxy)ethylidene]-α-D-glucopyranose) targets Shiga toxin through the trisaccharide moiety and the moiety comprising the cyclic pyruvate of glycerol targets SAP.

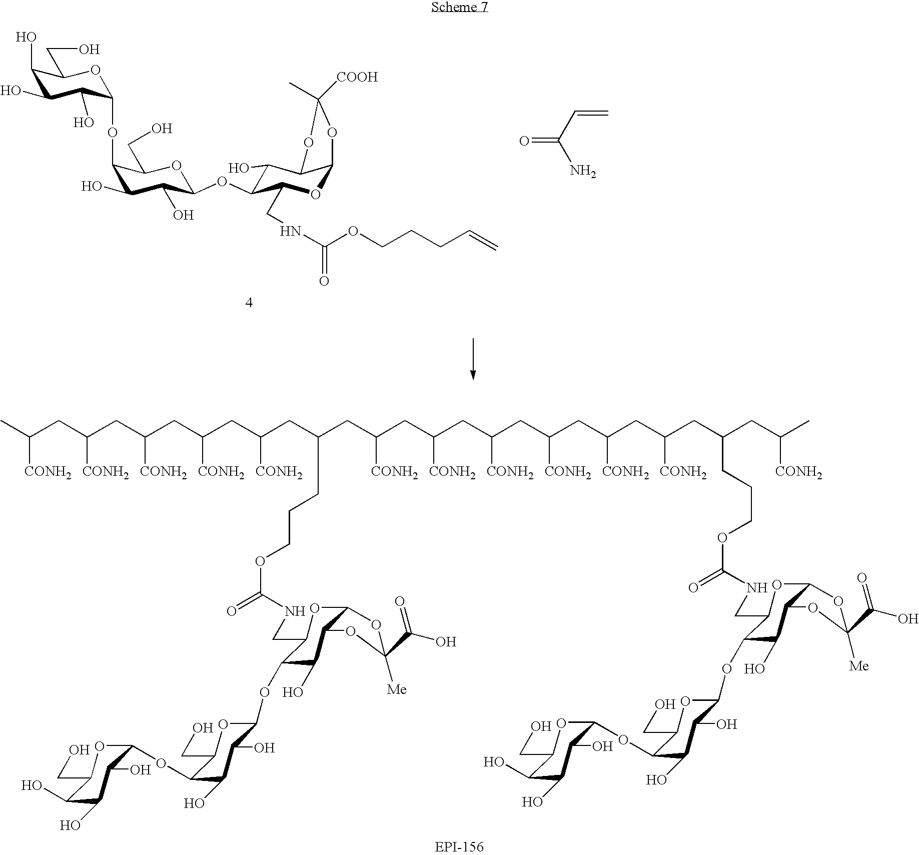

To a solution of acrylamide (34.2 mg, 0.5 mmol) and monomer 4 (66 mg, 0.096 mmol) in degassed water (1 mL) a solution of sodium persulfate (1 mg) in of water (10 μL) was added. The solution was spurged with argon and TEMED (12 μL) was added. The mixture was incubated for 16 h then dialyzed and freezed dried to give 26 mg of EPI-156. NMR data (not shown) indicate ˜4% of sugar monomer incorporation.

### Example 8

### Synthesis of 6-O-(2,5-dinitra-7-oxa-6-oxo-dodec-11-enoyl)-1,2-O—[(R)-1-(carboxy)ethylidene]-4-O-[4-O-(α-D-galactopyranosyl)-β-D-galactopyranosyl]-β-D-glucopyranose (23)

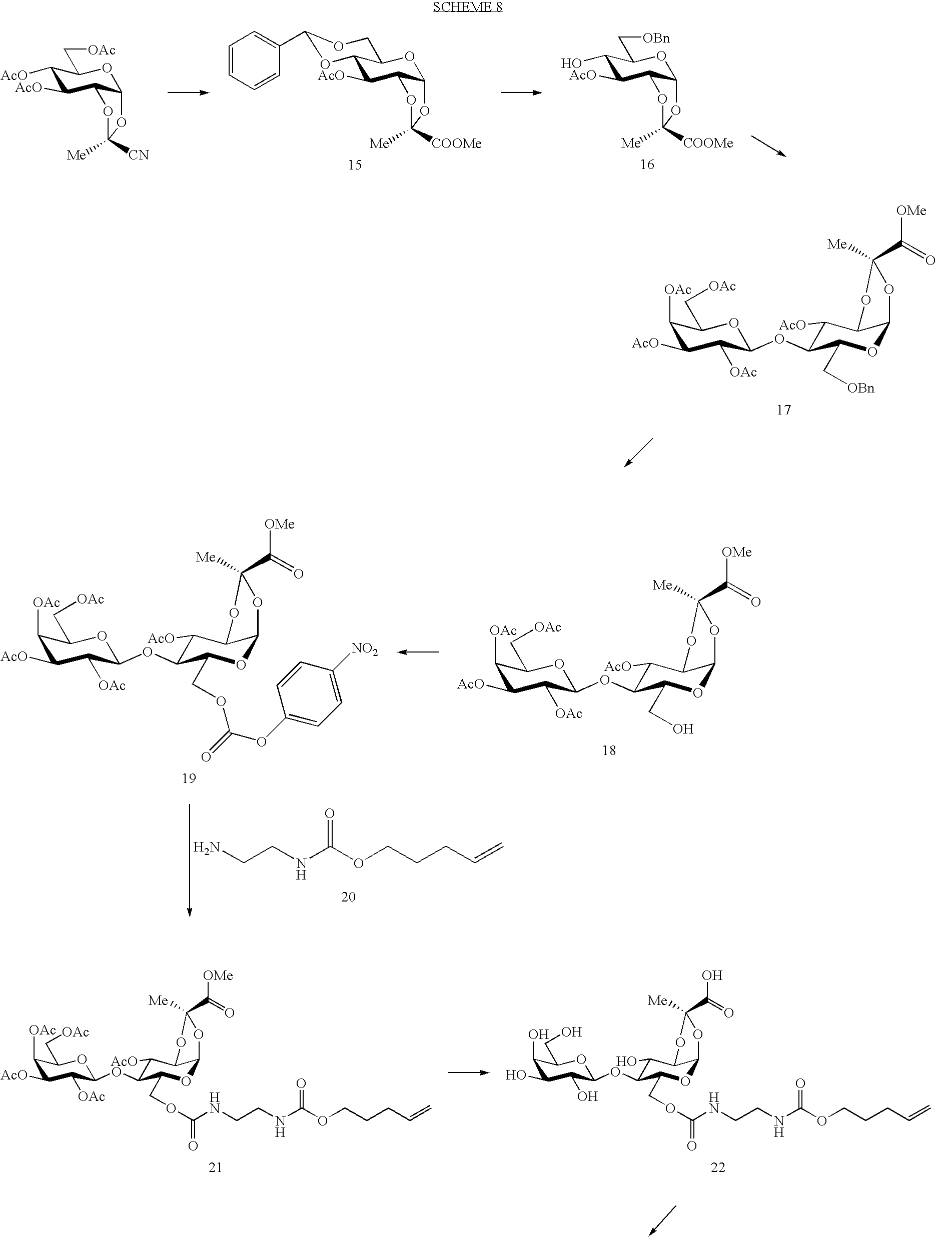

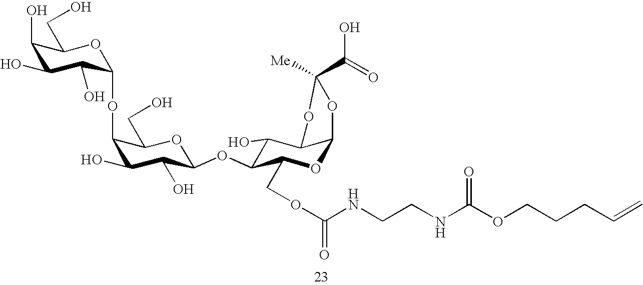

3-O-Acetyl-4,6-O-benzylidene-1,2-O—[(S)-1-(methoxycarbonyl)ethylidene]-α-D-glucopyranoside (15). To a solution of dry 1,2-O—[(S)-1-(methoxycarbonyl)ethylidene]-α-D-glucopyranoside (4.62 g, 15.1 mmol) in MeCN (30 mL), PhCH(OMe)₂ (2.27 mL, 1 eq) was added followed by CSA (100 mg). After 30 min, Py-Ac₂O (1:1 v/v, 10 mL) was added and the mixture was stirred for 3 h at RT. The reaction was quenched by addition of MeOH, concentrated and co-evaporated twice with toluene. Chromatography of the residue on silica gel (hexane:ethyl acetate (30-40%)) gave the title product (3.19 g, 54%), [α]_{D}+23° (c 1.2, CHCl₃). ¹H-NMR (CDCl₃): δ_{H} 7.5-7.35 (m, 5H, arom.), 5.84 (d, 1H, J_{1,2} 5.1 Hz, H-1), 5.53 (s, 1H, CHPh), 5.24 (dd, 1H, J_{2,3} 3.4 Hz, J_{3,4} 8.3 Hz, H-3), 4.41 (dd, 1H, J_{5,6a} 5.1 Hz, J_{6a,6b} 10.5 Hz, H-6a), 4.32 (dd, 1H, H-2), 3.94 (dd, 1H, J_{6b,5} 5.2 Hz, H-6b), 3.77-3.70 (m, 5H, H-4, H-5, CH₃), 2.126 (s, 3H, OAc), 1.767 (s, 3H, CH₃). ¹³C-NMR (CDCl₃): δ 169.79 (C═O), 169.48 (C═O), 136.80 (C arom.), 129.17 (CH arom.), 128.28 (CH arom.), 126.14 (CH arom.), 104.03 (C pyruvate), 101.59 (CH benzylidene), 98.84 (C-1), 77.68, 77.15, 73.07, 68.84 (C-6), 62.34, 52.69 (OCH₃), 22.39 (CH₃), 20.98 (CH₃). Electrospray ionization MS m/z 419.13136 ([M+Na]⁺, C₁₉H₂₄O₉Na⁺ requires 419.13125). Calculated for C₁₉H₂₄O₉: C, 57.86%; H, 5.62%. Found: C, 57.84%; H, 5.59%.

3-O-Acetyl-6-O-benzyl-1,2-O—[(S)-1-(methoxycarbonyl)ethylidene]-α-D-glucopyranoside (16). To a suspension of benzylidene derivative 15 (1.6 g, 4 mmol) and molecular sieves (4 Å, 1 g) in dry THF (30 mL), NaCNBH₃ (3.8 g, 60 mmol) was added followed by HCl-ether solution (2 M, ˜10 mL) until gas stopped to evolve. The mixture was neutralized by saturated aqueous NaHCO₃, filtered through celite, concentrated, then taken up in DCM, washed with water and concentrated. Chromatography of the residue on silica gel (hexane:ethyl acetate=1:1) gave the title compound 16 (1.16 g, 73%), [α]_{D}+13° (c 0.9, CHCl₃). ¹H-NMR (CDCl₃): δ_{H} 7.28-7.25 (m, 5 H, arom.), 5.82 (d, 1H, J_{1,2} 5.1 Hz, H-1), 5.04 (t, 1H, J_{2,3}˜J_{3,4}=3.4 Hz, H-3), 4.63 (d, 1H, J_{gem} 12.2 Hz, CH₂), 4.58 (d, 1H, CH₂), 4.39 (m, 1H, H-2), 3.84 (m, 1H, H-5), 3.79-3.75 (m, 5H, H-4, H-6a, CH₃), 3.72 (dd, 1H, J_{5,6b} 3.8 Hz, J_{6a,6b} 10.4 Hz, H-6b), 2.78 (s, 1H, OH), 2.11 (s, 3H, OAc), 1.75 (s, 3H, CH₃). ¹³C-NMR (CDCl₃): δ 170.72 (C═O), 169.42 (C═O), 137.74 (C arom.), 128.43 (CH arom.), 127.77 (CH arom.), 104.87 (C pyruvate), 98.07 (C-1), 74.82, 74.51, 73.67 (CH₂-Bn), 70.22, 69.62 (C-6), 69.23, 52.68 (OCH₃), 21.69 (CH₃), 20.88 (CH₃). Electrospray ionization MS m/z 417.11580 ([M+Na]⁺, C₁₉H₂₂O₉Na⁺ requires 417.11560). Calculated for C₁₉H₂₂O₉: C, 57.57%; H, 6.10%. Found: C, 57.42%; H, 6.01%.

### 3-O-Acetyl-4-O-(2,3,4,6-tetra-O-acetyl-β-D-galactopyranosyl)-6-O-benzyl-1,2-O—[(S)-1-(methoxycarbonyl)ethylidene]-α-D-glucopyranoside (17)

Tetra-O-acetyl-galactopyranose trichloroacetimidate (3.83 g, 7.77 mmol) and glycosyl acceptor 16 (2.77 g, 7 mmol) were combined, dried and dissolved in DCM (30 mL), then molecular sieves (4 Å, 1 g) were added. After 30 min, TMSOTf (100 μL) was added. After 1 h, the mixture was quenched with Py and concentrated. Chromatography of the residue on silica gel (hexane:ethyl acetate=1:1) gave 17 (2.66 g, 52%), [α]_{D}+4° (c 2.6, CHCl₃). ¹H-NMR (CDCl₃): δ_{H} 7.40-7.30 (m, 5H, arom.), 5.80 (d, 1H, J_{1,2} 5.2 Hz, H-1), 5.44 (dd, 1H, J_{2,3} 2.2 Hz, J_{3,4} 2.7 Hz, H-3), 5.34 (dd, 1H, J_{3′,4′} 3.5 Hz, J_{4′,5′} 1.0 Hz, H-4′), 5.12 (dd, 1H, J_{2′,3}^{,} 7.9 Hz, J_{3′,4′} 10.4 Hz, H-2′), 4.92 (dd, 1 H, H-3′), 4.70 (d, 1H, J_{gem} 12.2 Hz, Bn), 4.50 (d, 1H, Bn), 4.43 (d, 1H, H-1′), 4.32 (m, 1H, H-2), 4.13-4.09 (m, 2H, H-6′a, H-6′b), 3.87-3.78 (m, 3H, H-4, H-5, H-5′), 3.76 (s, 3H, CH₃), 3.67 (dd, 1H, J_{5,6a} 2.2 Hz, J_{6a,6b} 10.9 Hz, H-6a), 3.59 (dd, 1H, J_{5,6b} 3.4 Hz, H-6b), 2.16, 2.08, 2.03, 1.97, 1.92 (5 s, 15H, OAc), 1.73 (s, 3H, CH₃). ¹³C-NMR (CDCl₃): δ 170.35 (C═O), 170.29 (C═O), 170.07 (C═O), 169.42 (C═O), 169.11 (C═O), 169.06 (C═O), 137.82 (C arom.), 128.54 (CH arom.), 127.96 (CH arom.), 105.38 (C-pyruvate), 102.13 (C-1′), 98.08 (C-1), 76.10, 74.09, 73.60 (CH₂-Ph), 70.88, 70.69, 70.43, 68.90, 68.81, 68.33 (C-6′), 66.87, 61.03 (C-6), 52.65 (OCH₃), 21.26 (CH₃), 20.88 (CH₃), 20.69 (CH₃), 20.65 (CH₃), 20.62 (CH₃), 20.57 (CH₃). Electrospray ionization MS m/z 749.22665 ([M+Na]⁺, C₃₃H₄₂O₁₈Na⁺ requires 749.22634). Anal. Calculated for C₃₃H₄₂O₁₈: C, 54.54%; H, 5.83%. Found: C, 54.23%; H, 5.73%.

3-O-Acetyl-4-O-(2,3,4,6-tetra-O-acetyl-β-D-galactopyranosyl)-1,2-O—[(S)-1-(methoxycarbonyl)ethylidene]-α-D-glucopyranoside (18). To a solution of 17 (1.6 g, 2.2 mmol) in MeOH (10 mL), a drop of water and Pd(OH)₂ (30 mg) were added. After 2 h of stirring under H₂ atmosphere, the mixture was filtered via Millipore membrane filter, concentrated and chromatographed on silica gel (hexane:acetone=1:1) to give 18 (1.0 g, 71%), [α]_{D}+8° (c 1.2, CHCl₃). ¹H-NMR (CDCl₃): δ_{H} 5.76 (d, 1H, J_{1,2} 5.2 Hz, H-1), 5.51 (dd, 1H, J_{2,3}˜J_{3,4} 2.3 Hz, H-3), 5.38 (dd, 1H, J_{3′,4′} 3.5 Hz, J_{4′,5′} 0.9 Hz, H-4′), 5.18 (dd, 1H, J_{2′,3′} 8.0 Hz, J_{3′,4′} 10.4 Hz, H-2′), 5.01 (dd, 1H, H-3′), 4.64 (d, 1H, H-1′), 4.33 (m, 1H, H-2), 4.14-4.10 (m, 2H, H-6′a, H-6′ b), 3.93 (td, 1H, J_{4′,5′} 0.9 Hz, J_{5′,6a′}˜J_{5′,6b′} 6.7 Hz, H-5′), 3.85-3.82 (m, 2H, H-4, H-6a), 3.766 (s, 3H, CH₃), 3.75 (m, 1H, H-5), 3.61 (dd, 1H, J_{5,6b} 3.8 Hz, J_{6a,6b} 12.0 Hz, H-6b), 2.16, 2.09, 2.06, 2.03, 1.98 (5 s, 15H, OAc), 1.74 (s, 3H, CH₃). ¹³C-NMR (CDCl₃): δ 170.37 (C═O), 170.27 (C═O), 170.07 (C═O), 169.32 (C═O), 169.08 (C═O), 105.47 (C-pyruvate), 102.25 (C-1′), 97.78 (C-1), 76.49, 74.40, 70.93, 70.84. 70.36, 69.05, 68.98, 66.89, 61.80 (C-6′), 61.05 (C-6), 52.69 (OCH₃), 21.27 (CH₃), 20.86 (CH₃), 20.68 (CH₃), 20.64 (CH₃), 20.55 (CH₃). Electrospray ionization MS m/z 659.17920 ([M+Na]⁺, C₂₆H₃₆O₁₈Na⁺ requires 659.17939). Calculated for C₂₆H₃₆O₁₈: C, 49.06%; H, 5.70%. Found: C, 49.12%; H, 5.70%.

3-O-Acetyl-6-O-(4-nitrophenyl)-4-O-(2,3,4,6-tetra-O-acetyl-β-D-galactopyranosyl)-1,2-O—[(S)-1-(methoxycarbonyl)ethylidene]-α-D-glucopyranoside (19). To a solution of 18 (1 g, 1.57 mmol) and 4-nitrophenyl chloroformate (380 mg, 1.2 eq) in dry DCM (10 mL), Py (˜0.2 mL) was added. TLC (hexane:acetone=1:1) shows complete conversion after 5 min Water (0.2 mL) was added and the mixture was concentrated and chromatographed on silica gel in hexane:ethyl acetate=1:1 to give 19 (1.2 g, 95%), [α]_{D}+0.7° (c 1, CHCl₃). ¹H-NMR (CDCl₃): δ_{H} 8.31-8.28 (m, 2H, arom.), 7.42-7.39 (m, 2H, arom.), 5.80 (d, 1H, J_{1,2} 5.1 Hz, H-1), 5.55 (dd, 1H, J_{2,3} 1.6 Hz, J_{3,4} 2.5 Hz, H-3), 5.39 (dd, 1H, J_{3′,4′} 3.5 Hz, J_{4′,5′} 1.1 Hz, H-4′), 5.21 (dd, 1H, J_{2′,3′} 8.0 Hz, J_{3′,4′} 10.4 Hz, H-2′), 5.04 (dd, 1H, H-3′), 4.70 (d, 1H, H-1′), 4.51 (dd, 1H, J_{6a,5} 2.3 Hz, J_{6a,6b} 11.7 Hz, H-6a), 4.39 (m, 1H, H-2), 4.51 (dd, 1H, J_{6b,5} 5.7 Hz, H-6b), 4.17 (dd, 1H, J_{6′a,5} 6.5 Hz, J_{6′a,6′b} 11.3 Hz, H-6a), 4.07 (m, 1H, H-5), 3.93 (td, 1H, J_{4′,5′} 1.1 Hz, J_{5′,6a′}˜J_{5′,6b′} 6.6 Hz, H-5′), 3.78 (m, 4H, H-4, CH₃), 2.17, 2.12, 2.07, 2.05, 1.98 (5 s, 15H, OAc), 1.78 (s, 3H, CH₃). ¹³C-NMR (CDCl₃): δ 170.35 (C═O), 170.21 (C═O), 170.05 (C═O), 169.38 (C═O), 169.09 (C═O), 169.03 (C═O), 155.33 (C—O of Ph), 152.28 (C═O of carbonate), 145.53 (C—NO₂ of Ph), 125.36 (CH of Ph), 121.71 (CH of Ph), 105.67 (C-pyruvate), 101.66 (C-1′), 97.65 (C-1), 77.26, 74.22, 71.00, 70.82. 69.71, 68.89, 67.76 (C-6), 66.85, 66.57, 61.18 (C-6′), 52.79 (OCH₃), 21.20 (CH₃), 20.84 (CH₃), 20.69 (CH₃), 20.67 (CH₃), 20.64 (CH₃), 20.54 (CH₃). Electrospray ionization MS m/z 824.18573 ([M+Na]⁺, C₃₃H₃₉NO₂₂Na⁺ requires 824.18559). Calculated for C₃₃H₃₉NO₂₂: C, 49.44%; H, 4.90%; N, 1.75%. Found: C, 49.64%; H, 4.88%; N, 2.07%.

2-(pent-4-enyloxycarbamoyl)-ethylamine (20). To a solution of pent-4-enol-1 (0.97 g, 11.26 mmol) and 4-nitrophenyl chloroformate (2.3 g) in dry DCM (7 mL), Py (0.92 mL) was slowly added. After 30 min, the resulting mixture was added to a solution of 1,2-diaminoethane (2 mL) in DCM (10 mL). After 20 min, the mixture was washed with brine and the DCM fractions were concentrated. Chromatography of the residue on silica gel (DCM-MeOH=1:1-0:1) gave the product 7 as a slightly yellow syrup (1.11 g; 57%). ¹H-NMR (CDCl₃): δ_{H} 5.88-5.74 (m, 1H, CH═CH₂), 5.15-4.90 (m, 3H, NH, CH═CH₂), 4.06 (t, 2H, J 6.6 Hz, CH₂O), 3.21 (t, 2H, J 6.0 Hz, CH₂NHCO), 2.81 (t, 2H, CH₂NH₂), 2.16-2.06 (m, 2H, CH₂CH═CH₂), 1.76-1.60 (m, 2H, CH₂CH₂CH₂). ¹³C-NMR (CDCl₃): δ 156.90 (C═O), 137.59 (CH═CH₂), 115.12 (CH═CH₂), 64.29 (CH₂O), 42.89 (CH₂N), 41.34 (CH₂N), 29.98 (CH₂), 28.24 (CH₂). Electrospray ionization MS m/z 173.12849 ([M+H]⁺, C₈H₁₇N₂O₂⁺ requires 173.12845).

3-O-acetyl-6-O-(2,5-dinitra-7-oxa-6-oxo-dodec-11-enoyl)-1,2-O—[(R)-1-(methoxycarbonyl)ethylidene]-4-O-(2,3,4,6-tetra-O-acetyl-β-D-galactopyranosyl)-α-D-glucopyranose (21). To a solution of 20 (250 mg) in DCM, a solution of 19 (884 mg, 1.1 mmol) was added followed by Et₃N (300 μL). After 1 h with occasional heating below boiling point, TLC (hexane:acetone=1:1) indicated completion of the reaction. The mixture was concentrated and the residue was chromatographed on silica gel (hexane:acetone=1:1) to give 21 (550 mg, 60%), [α]_{D}+0.8° (c 1, CHCl₃). ¹H-NMR (CDCl₃): δ_{H} 5.84-5.76 (m, 1H, —CH═CH₂), 5.76 (d, 1H, J_{1,2} 5.2 Hz, H-1), 5.53 (dd, 1H, J_{2,3} 1.4 Hz, J_{3,4} 2.3 Hz, H-3), 5.38 (dd, 1H, J_{3′,4′} 3.5 Hz, J_{4′,5′} 0.9 Hz, H-4′), 5.23 (broad s, 1H, NH), 5.18 (dd, 1H, J_{2′,3′}8.2 Hz, J_{3′,4′} 10.4 Hz, H-2′), 5.08-4.97 (m, 4H, H-3′, NH, —CH═CH₂), 4.63 (d, 1H, H-1′), 4.35 (m, 1H, H-2), 4.22 (dd, 1H, J_{6a,5} 2.0 Hz, J_{6a,6b} 11.6 Hz, H-6a), 4.18-4.09 (m, 3H, H-6b, H-6′a, H-6′b), 4.07 (m, 2H, CH₂), 3.95 (td, 1H, J_{4′,5′} 1.0 Hz, J_{5′,6a′}˜J_{5′,6b′}=7.3 Hz, H-5′), 3.90 (m, 1H, H-5), 3.77 (s, 3H, CH₃), 3.64 (d, 1H, H-4), 3.32 (broad s, 4H, CH₂N), 2.17, 2.10, 2.08, 2.03, 1.98 (5 s, 15H, OAc), 2.14-2.10 (m, 2H, CH₂), 1.75 (s, 3H, CH₃), 1.74-1.68 (m, 2H, CH₂). ¹³C-NMR (CDCl₃): δ 170.37 (C═O), 170.28 (C═O), 170.07 (C═O), 169.49 (C═O), 169.25 (C═O), 169.01 (C═O), 157.03 (C═O), 156.27 (C═O), 137.53 (—CH═CH₂), 115.20 (—CH═CH₂), 105.64 (C-pyruvate), 102.37 (C-1′), 97.71 (C-1), 77.32, 73.93, 70.88, 70.79, 69.84, 68.89, 67.21, 66.87, 64.50 (CH₂), 64.12 (CH₂), 61.99 (CH₂), 52.69 (OCH₃), 41.31 (CH₂), 41.06 (CH₂), 30.00 (CH₂), 28.19 (CH₂), 21.15 (CH₃), 20.86 (CH₃), 20.70 (CH₃), 20.66 (CH₃), 20.55 (CH₃). Electrospray ionization MS m/z 857.27929 ([M+Na]⁺, C₃₅H₅₀N₂O₂₁Na⁺ requires 857.27983). Calculated for C₃₅H₅₀N₂O₂₁: C, 50.36%; H, 6.04%, N, 3.36%. Found: C, 50.37%; H, 6.15%; N, 3.35%.

6-O-(2,5-dinitra-7-oxa-6-oxo-dodec-11-enoyl)-1,2-O—[(R)-1-(carboxy)ethylidene]-4-O-β-D-galactopyranosyl)-α-D-glucopyranose (22). The protected derivative 21 (0.53 g, 0.63 mmol) was dissolved in dry MeOH (3.5 mL) and NaOMe (1 M, 0.64 mL) in MeOH was added. The mixture was stirred at room temperature for 2 h then concentrated and the resulting solid was dissolved in water (3 mL). After 1 h, the hydrolysis of the methyl ester was complete. The solution was neutralized with acetic acid, concentrated used directly in the next step. A small sample was purified by HPLC chromatography on C-18 in water-MeOH containing 1% AcOH, [α]_{D}+19° (c 1, H₂O). ¹H NMR (D₂O) δ: 5.90 (m, 1H, CH═CH₂), 5.62 (d, 1H, J_{1,2} 4.9 Hz, H-1), 5.10-5.01 (m, 2H, CH═CH₂), 4.44 (d, 1H, J_{1′,2′} 7.8 Hz, H-1′), 4.41-4.34 (m, 2H, H-3, H-6a), 4.23 (dd, 1H, J_{5,6b} 5.3 Hz, J_{6a,6b} 12.0 Hz, H-6b), 4.18 (m, 1H, H-2), 4.08-4.01 (m, 3H, H-5, OCH₂), 3.92 (d, 1H, J_{3′,4′} 3.4 Hz, H-4′), 3.83-3.74 (m, 3H, H-4, H-6′a, H-6′b), 3.69 (m, 1H, H-5′), 3.64 (dd, 1H, J_{2′,3′} 9.9 Hz, H-3′), 3.55 (m, 1H, H-2′), 3.24 (s, 4H, NCH₂), 2.12 (m, 2H, CH₂), 1.72 (m, 2H, CH₂), 1.64 (s, 3H, CH₃). ¹³C-NMR (CDCl₃): δ 181.28 (C-0), 159.23 (C═O), 158.41 (C═O), 138.85 (—CH═CH₂), 115.26 (—CH═CH₂), 107.36 (C-pyruvate), 105.15 (C-1′), 96.74 (C-1), 78.53, 75.61, 75.37, 72.84, 71.04, 69.54, 68.87, 68.52, 65.29 (CH₂), 64.34 (CH₂), 61.33 (CH₂), 40.56 (CH₂), 40.35 (CH₂), 29.66 (CH₂), 27.75 (CH₂), 21.76 (CH₃). Electrospray ionization MS m/z 609.21365 ([M]⁻), C₂₄H₃₇N₂O₁₆⁻ requires 609.21376). Calculated for C₂₄H₃₈N₂O₁₆: C, 47.21%; H, 6.27%; N, 4.59%. Found: C, 46.67%; H, 6.23%; N, 4.57%.

6-O-(2,5-dinitra-7-oxa-6-oxo-dodec-11-enoyl)-1,2-O—[(R)-1-(carboxy)ethylidene]-4-O-[4-O-(α-D-galactopyranosyl)-β-D-galactopyranosyl]-α-D-glucopyranose (23). Deprotected lactose derivative 22 was dissolved in HEPES buffer [2 mL, 1.6 M, 10 mM MnCl₂, bovine serum albumin (BSA, 0.8 mg/mL), water (7.14 mL) followed by the addition of alkaline phosphatase (54 μL) and UDP-glucose (0.58 g; 1.5 eq.). α-(1,4)-galactosyltransferase/UDP-4″-Gal-epimerase (0.7 mL) was added to the reaction mixture and it was incubated at 37° C. After 18 h, NMR indicated that the reaction was complete. The reaction mixture was concentrated. The residue was treated with methanol, the solid precipitate was filtered off and rinsed with methanol. The combined methanol solution fractions were concentrated and chromatographed on silica gel (DCM-MeOH (4% of AcOH)=6:4-4:5). The product was dissolved in water, filtered through 0.2 μm membrane and freeze-dried to give white powder 23 (394 mg; 80%), [α]_{D}+61° (c 1, H₂O). ¹H-NMR (D₂O): δ_{H} 5.90 (m, 1H, CH═CH₂), 5.73 (d, 1H, J_{1,2} 4.9 Hz, H-1), 5.10-5.02 (m, 2H, CH═CH₂), 4.94 (d, 1H, J_{1″,2″} 4.1 Hz, H-1″), 4.51 (d, 1H, J_{1′,2′} 7.7 Hz, H-1′), 4.42-4.36 (m, 3H, H-3, H-6a, H-5″), 4.31 (t, 1H, J_{2,3} 4.0 Hz, H-2), 4.24 (dd, 1H, J_{5,6b} 5.4 Hz, J_{6a,6b} 12.0 Hz, H-6b), 4.07 (t, 2H, J 6.5 Hz, OCH₂), 4.04-4.01 (m, 3H, H-4′, H-4″, H-5), 3.94-3.90 (m, 2H, H-5′, H-3″), 3.86-3.68 (m, 7H, H-4, H-3′, H-6a′, H-6b′, H-2″, H-6a″, H-6b″), 3.58 (dd, 1H, J_{2′,3′} 10.3 Hz, H-2′), 3.24 (bs, 4H, NCH₂), 2.14 (m, 2H, CH₂), 1.73 (m, 5H, CH₂, CH₃), 1.65 (s, 3H, CH₃). ¹³C-NMR (D₂O): δ 174.53 (C═O), 159.85 (C═O), 159.00 (C═O), 139.44 (—CH═CH₂), 115.84 (—CH═CH₂), 106.71 (C-pyruvate), 106.10 (C-1″), 101.13 (C-1′), 97.82 (C-1), 79.44, 77.89, 76.38, 76.10, 73.01, 71.62, 70.06, 69.87, 69.60, 69.30, 65.90 (CH₂), 64.99 (CH₂), 61.39 (CH₂), 61.12 (CH₂), 41.15 (CH₂), 40.96 (CH₂), 30.26 (CH₂), 28.36 (CH₂), 21.85 (CH₃). Electrospray ionization MS m/z 795.26438 ([M+Na]⁺), C₃₀H₄₈N₂O₂₁Na⁺ requires 795.26418. Calculated for C₃₀H₄₇N₂NaO₂₁: C, 45.34%; H, 5.96%; N, 3.53%. Found: C, 45.38%; H, 6.11%; N, 3.80%.

### Example 9

### Synthesis of a Co-Polymer of 23 with Acrylamide (EPI-29)

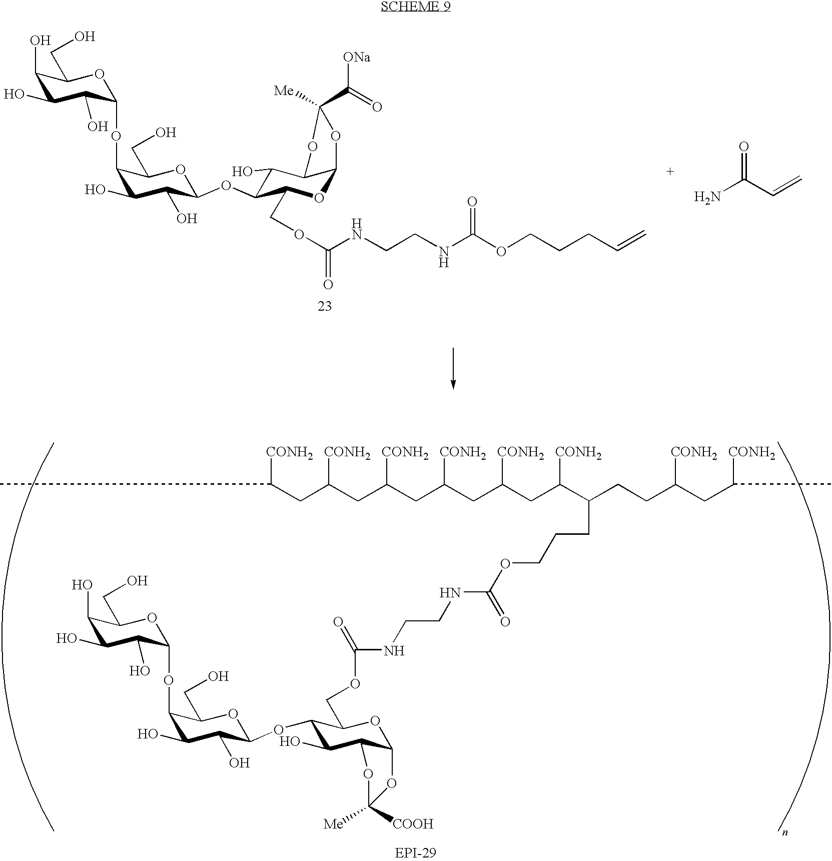

The solution of 23 (471 mg; 0.61 mmol) and acrylamide (264 mg; 3.7 mmol) in Tris buffer (0.2 M, pH 9; 7.32 mL) was sparged with argon, then a solution of ammonium persulfate (7.43 mg) in Tris buffer (74.3 μL) was added and sparged with argon. TEMED (37.15 μL) was added to the reaction mixture and it was vortexed briefly then left at room temperature overnight. The mixture was diluted with ethanol (40 mL). The precipitate was collected, washed with ethanol, taken up in water and dialyzed 4 times against deionized water (2 L). The dialyzed solution of the polymer was filtered through 0.2 μm membrane and freeze-dried to provide the product EPI-29 as a white powder (390 mg). NMR indicated 5 molar % ligand incorporation.

### Example 10

### Synthesis of methacrylate monomer {2-{1,2-O—[(R)-1-(carboxy)ethylidene]-4-O-[4-O-(α-D-galactopyranosyl)-β-D-galactopyranosyl]-α-D-glucopyranose}-6-yloxycarbonylamino}-carbamic acid 5-[2-(2-methyl-acryloylamino)-ethylsulfanyl]-pentyl ester (25)

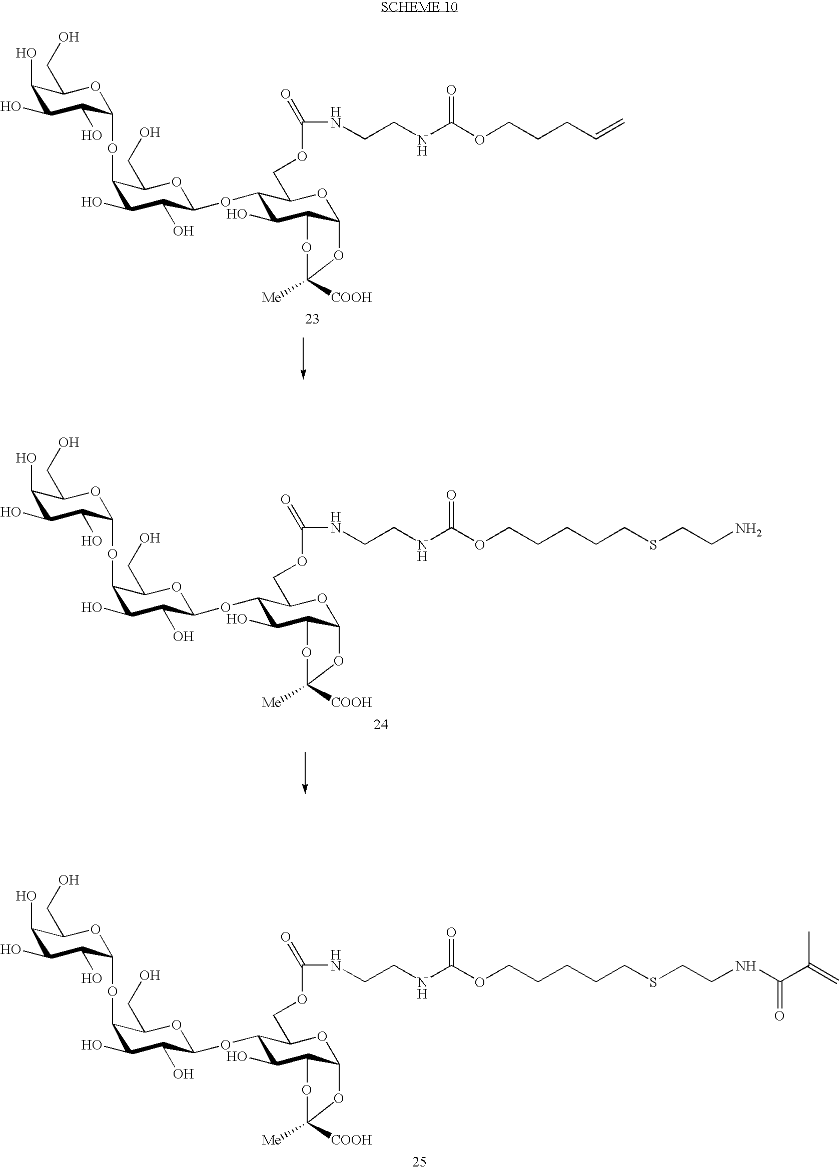

{2-{1,2-O—[(R)-1-(carboxy)ethylidene]-4-O-[4-O-(α-D-galactopyranosyl)-β-D-galactopyranosyl]-β-D-glucopyranose}-6-yloxycarbonylamino}-ethyl]-carbamic acid 5-(2-amino-ethylsulfanyl)-pentyl ester (24). Compound 23 (91 mg, 0.107 mmol) was dissolved in degassed water (3 mL) in a quartz tube and purged with argon, then cysteamine hydrochloride (244 mg, 2.14 mmol) was added to the solution and the mixture was sparged with argon. It was irradiated under argon with UV light (Ray-Pen) for 15 min cycles (5 times) with short break to cool the UV lamp. The product of the reaction was purified on reversed phase silica gel C18 using water-methanol with 0.1% TFA. After concentration and freeze-drying, product 24 was obtained as a white foam (94 mg, 100%), [α]_{D}+44° (c1.12, water), ¹H-NMR (D₂O): δ 5.74 (d, 1 H, J_{1,2} 4.95 Hz, H-1), 4.96 (d, 1H, J_{1″,2″} 3.94 Hz, H-1″), 4.52 (d, 1H, J_{1′,2′} 7.8 Hz, H-1′), 4.42-4.37 (m, 3H, J_{6a,6b} 11.9 Hz, H-3, H-6a, H-5″), 4.32 (dd, 1H, J_{2,3} 4.0 Hz, H-3), 4.25 (dd, 1H, J_{5,6b} 5.6 Hz, H-6b), 4.12-4.00 (m, 5H, H-5, H-4′, H-4″, OCH₂), 3.96-3.68 (m, 9H, H-4, H-3′, H-5′, H-6a′, H-6b′, H-2″, H-3″, H-6a″, H-6b″), 3.60 (dd, 1H, H-2′), 3.27-3.20 (m, 6H, NCH₂), 2.85 (t, 2H, J 6.7 Hz, SCH₂), 2.61 (t, 2H, J 7.3, SCH₂, 1.55 (s, 3H, CH₃), 1.54-1.52 (m, 4H, CH₂), 1.50-1.48 (m, 3H, CH₂). ¹³C-NMR (D₂O): δ 174.27 (C═O), 159.86 (C═O), 158.99 (C═O), 106.54 (C-pyruvate), 106.09 (C-1″), 101.13 (C-1′), 97.88 (C-1), 79.43, 77.71, 76.39, 76.16, 73.03, 71.63, 70.07, 69.88, 69.62, 69.59, 69.35, 66.34 (CH₂), 65.00 (CH₂), 61.41 (CH₂), 61.15 (CH₂), 41.18 (CH₂), 41.17 (CH₂), 39.27 (CH₂), 31.45 (CH₂), 29.06 (CH₂), 29.01 (CH₂), 28.69 (CH₂), 25.14 (CH₂), 21.85 (CH₃). HRMS-ES m/z 872.29460 [M+Na]⁺, C₃₂H₅₅N₃O₂₁SNa⁺ requires 872.29410.

{2-{1,2-O—[(R)-1-(carboxy)ethylidene]-4-O-[4-O-(α-D-galactopyranosyl)-β-D-galactopyranosyl]-β-D-glucopyranose}-6-yloxycarbonylamino}-carbamic acid 5-[2-(2-methyl-acryloylamino)-ethylsulfanyl]-pentyl ester (25). The cysteamine adduct 24 (38 mg, 0.44 mmol) was dissolved in water (3 mL) and N-methacryloxysuccinimide (30 mg, 0.16 mmol) was added followed by dry sodium bicarbonate until pH 8 was reached. The mixture was stirred at room temperature for 1 h, then acidified with 5M acidic acid. Chromatography on reversed phase silica gel using water-methanol with 1% of acetic acid provided pure product 25 as a white foam after freeze-drying (28 mg, 69%), [α]_{D}+43.7° (c1.24, water), ¹H-NMR (D₂O): δ 5.71 m (s, 1H, C═CH₂), 5.69 (d, 1H, J_{1,2} 4.8 Hz, H-1), 5.46 (s, 1H, C═CH₂), 4.95 (d, 1H, J_{1′,2″} 3.9 Hz, H-1″), 4.52 (d, 1H, J_{1′,2′} 7.7 Hz, H-1′), 4.42-4.36 (m, 3H, H-3, H-6a, H-5″), 4.30-4.21 (m, 2 H, H-2, H-6b), 4.10-4.00 (m, 3H, H-5, H-4′, H-4″), 3.94-3.90 (m, 2H, H-5, H-3″), 3.86-3.76 (m, 4H, H-6a′, H-6b′, H-2″, H-6a″), 3.74-3.67 (m 3H, H-4, H-3′, H-6b″), 3.59 (dd, 1H, J_{2′3}, 7.9 Hz, H-2′), 3.47 (t, J 6.0 Hz, NCH₂), 3.24 (bs, 4H, NCH₂), 2.75 (t, 2H, J 6.7 Hz, SCH₂), 2.61 (t, 2H, J J 7.1 Hz, SCH₂), 1.93 (s, 3H, CH₃), 1.69 (s, 3H, CH₃), 1.66-1.58 (m, 4H, CH₂), 1.50-1.40 (m, 2H, CH₂). ¹³C-NMR (D₂O): δ 174.59 (C═O), 159.78 (C═O), 158.91 (C═O), 139.88 (H₂C═CCH₃), 121.96 (H₂C═CCH₃), 106.76 (C-pyruvate), 106.6 (C-1″), 101.08 (C-1′), 97.74 (C-1), 79.42, 77.84, 76.32, 76.05, 72.97, 71.57, 70.01, 69.82, 69.58, 69.54, 69.22, 66.33 (CH₂), 66.32 (CH₂), 64.95 (CH₂), 61.39 CH₂), 61.34 (CH₂), 61.12 (CH₂), 61.07 (CH₂), 41.11 (CH₂), 39.82 (CH₂), 31.85 (CH₂), 31.16 (CH₂), 29.17 (CH₂), 28.62 (CH₂), 25.15 (CH₂), 21.85 (CH₃), 18.53, 18.51 (CH₃). HRMS-ES m/z 916.32261 [M−H], C₃₆H₅₈N₃O₂₂S requires 916.32382.

### Example 11

### Synthesis of a Co-Polymer of Compound 25 with HPMA (HPMA-B2)

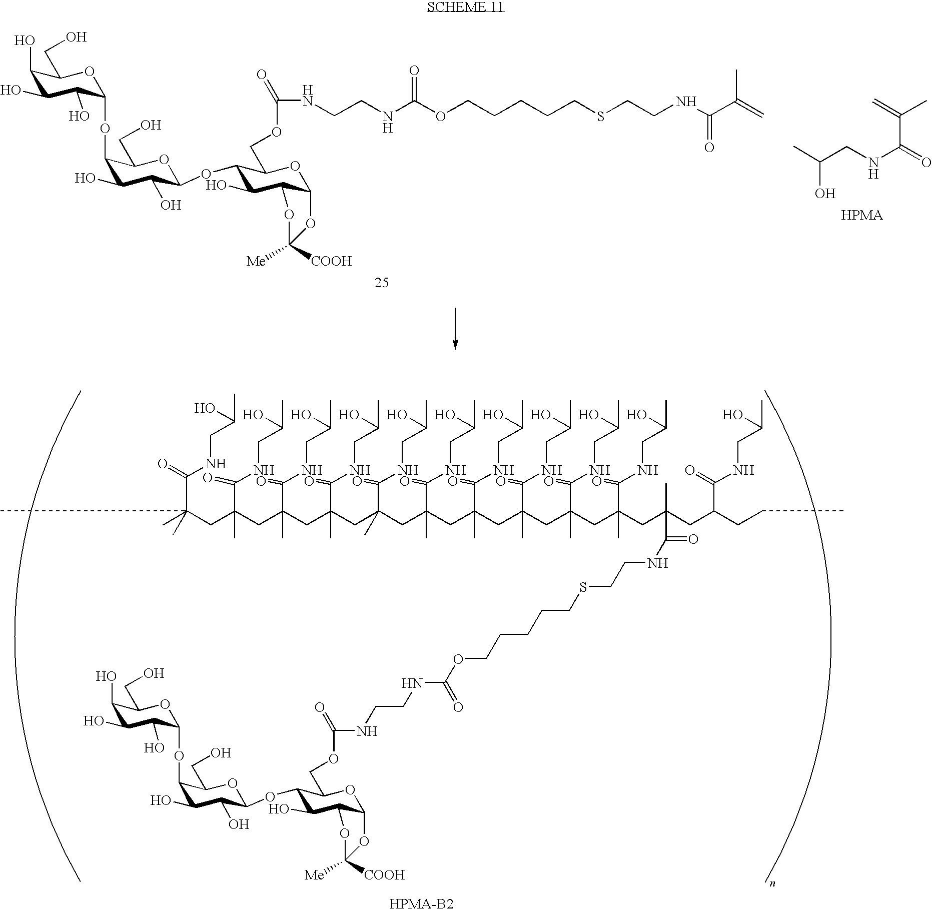

Compound 25 (27 mg, 0.03 mmol) and HPMA monomer (72 mg, 0.5 mmol) were dissolved in degassed acetate buffer (0.1 M, pH 4). The solution was sonicated under vacuum and then saturated with argon. A solution of ammonium persulfate (2 mg in 20 μL of the acetate buffer) was added to the mixture and it was sonicated under vacuum and saturated with argon. Finally, cysteamine solution (0.14 mg in 28 μL of the acetate buffer) was added to the reaction mixture and, after vortexing for a few seconds, it was incubated at 50° C. overnight. After 21 h, the solution was treated with acetone (8 mL). The precipitate was separated then dissolved in water and dialyzed against deionized water, then filtered through 0.45 μm membrane and freeze-dried to provide product as a white powder (25 mg). ¹H-NMR in D₂O indicates 5 molar % incorporation of the ligand into HPMA-B2 polymer.

### Example 12

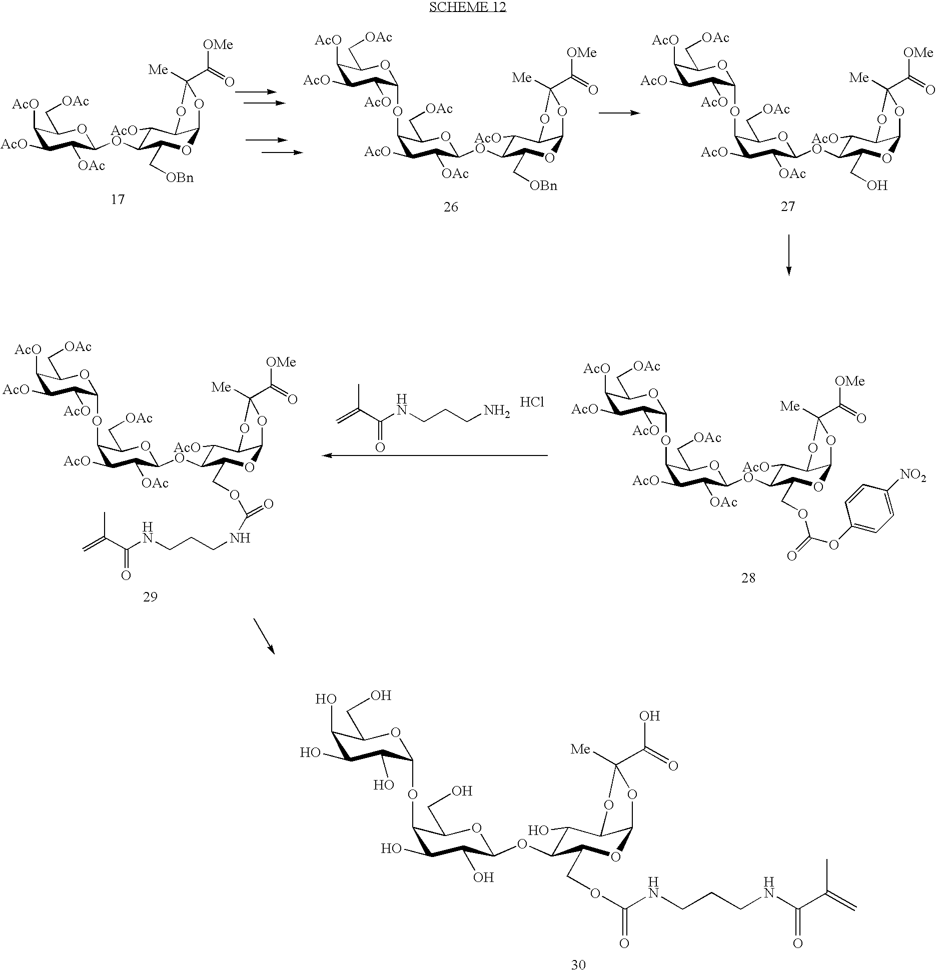

3-O-Acetyl-6-O-benzyl-1,2-O—[(R)-1-(methoxycarbonyl)ethylidene]-4-O-[2,3,6-tri-O-acetyl-4-O-(2,3,4,6-tetra-O-acetyl-α-D-galactopyranosyl)-β-D-galactopyranosyl]-α-D-glucopyranose (26). The acetate 17 (0.973 g; 1.34 mmol) was dissolved in dry methanol (2.5 mL) and 0.5 M NaOMe (2.8 mL) was added. After 1 hr at room temperature the mixture was treated with Dowex H+ resin, filtered and concentrated to provide white foam. The product (0.764 g) was dissolved in water (15 mL) and HEPES buffer was added (4.58 mL) followed by DTT solution (1.14 mL) and alkaline phosphatase (114 μL). Then UDP-Glc (1.23 g) was added to the mixture followed by fused enzyme (1.5 mL). The reaction was incubated overnight at 37° C. Then the mixture was diluted with water, acidified with Dowex H+ resin, filtered and the supernatant was concentrated. To a suspension of the residue in dry methanol (25 mL) acetyl chloride (0.25 mL) was added and the mixture was stirred at room temperature for 3.5 h then neutralized with pyridine. The solid residue was filtered off and rinsed with methanol, the filtrate was concentrated and acetylated (10 mL of pyridine, 10 mL of acetic anhydride, 48 h). A few drops of methanol were added to the mixture to quench the excess of the reagent and the mixture was concentrated and co-evaporated with toluene. The crude product was purified on silica gel column using hexane-acetone (7:3-2:1) to provide pure title product as a white foam (0.814 g; 60%). NMR (CDCl₃): δ 7.39-7.29 (m, 5H, C₆H₅), 5.80 (d, 1H, J_{1,2} 5.2 Hz, H-1), 5.60 (dd, 1H, J_{3″,4″} 3.4 Hz, J_{4″,5″} 1.3 Hz, H-4″), 5.49 (t, 1H, J_{2,3}=J_{3,4}=2.6 Hz, H-3), 5.37 (dd, 1H, J_{2″,3″} 11.0 Hz, J_{3″,4″} 3.4 Hz, H-3″), 5.19, (dd, 1H, J_{1″,2″} 3.7 Hz, H-2″), 5.13 (dd, 1H, J_{1′,2′} 7.8 Hz, J_{2′,3′} 11.8 Hz, H-2′), 5.02 (d, 1H, H-1″), 4.76 (dd, 1H, J_{3′,4′} 2.9 Hz, H-3′), 4.69 (d, 1H, J 12.2 Hz, CH₂), 4.54-4.49 (m, 3H, J_{5″,6a″} 8.4 Hz, H-1′, H-5″, CH₂), 4.37 (dd, 1H, 6.5 Hz, J_{6a′,6b′} 11.2 Hz, H-6a), 4.32 (dd, 1H, H-2), 4.16 (dd, 1H, J_{6a″,6b″} 10.9 Hz, H-6a″), 4.11-4.06 (m, 2H, H-6b′, H-6b″), 4.03 (d, 1H, H-4′), 3.89-3.83 (m, 2H, H-4, H-5′), 3.76 (s, 3H, OCH₃), 3.73 (dd, 1H, H-5′), 3.69 (dd, 1H, H6a), 3.63 (dd, 1H, H-6b), 2.13, (s, 3H, CH₃), 2.11 (s, 3H, CH₃), 2.09 (s, 3H, CH₃), 2.07 (s, 3H, CH₃), 2.05 (s, 3H, CH₃), 2.04 (s, 3H, CH₃), 1.99 (s, 3H, CH₃), 1.92 (s, 3H, CH₃), 1.75 (s, 3H, CH₃). HRMS-ES m/z 1037.31122 [M+Na]⁺, C₄₅H₅₈O₂₆Na⁺ requires 1037.31085.

3-O-Acetyl-1,2-O—[(R)-1-(methoxycarbonyl)ethylidene]-4-O-[2,3,6-tri-O-acetyl-4-O-(2,3,4,6-tetra-O-acetyl-α-D-galactopyranosyl)-β-D-galactopyranosyl]-α-D-glucopyranose (27). Compound 26 (0.804 g; 0.79 mmol) was dissolved in methanol (5 mL) and hydrogenated in the presence of 10% palladium on carbon. After 2 h the mixture was diluted with methanol. The catalyst was filtered off and rinsed with methanol. The filtrate was concentrated to provide pure product as a white solid (0.73 g; 100%). NMR (CDCl₃): δ 5.77 (d, 1H, J_{1,2} 5.1 Hz, H-1), 5.60 (dd, 1H, J_{3″,4″} 3.4 Hz, J_{4″,5″} 1.1 Hz, H-4″), 5.49 (t, 1H, J_{2,3}=J_{3,4} 2.4 Hz, H-3), 5.38 (dd, 1H, J_{2″,3″} 11.0 Hz, H-3″), 5.22-5.17 (m, 2H, J_{1′,2′} 7.9 Hz, J_{2′,3′} 10.8 Hz, J_{1″,2″} 3.8 Hz, H-2′, H-2″), 5.01 (d, 1H, H-1″), 4.86 (dd, 1H, J_{3′,4′} 2.8 Hz, H-3′), 4.67 (d, 1H, H-1′), 4.56 (m, 1H, H-5″), 4.40 (dd, 1H, J_{5′,6a′} 6.5 Hz, J_{6a′,6b′} 11.2 Hz, H-6a′), 4.35-4.33 (m, 1H, H-2), 4.19-4.05 (m, 4H, H-4′, H-6b′, H-6a″, H-6b″), 4.89-4.78 (m, 4H, H-4, H-5, H-6a, H-5′), 4.77 (s, 3H, OCH₃), 4.68-4.62 (m, 1H, H-6b), 2.14 (s, 3H, CH₃), 2.12 (s, 3H, CH₃), 2.11 (s, 3H, CH₃), 2.08 (s, 3H, CH₃), 2.07 (s, 3H, CH₃), 2.06 (s, 3H, CH₃), 2.04 (s 3H, CH₃), 2.00 (s, 3H, CH₃), 1.76 (s, 3H, CH₃). HRMS-ES m/z 947.26346 [M+Na]⁺, C₃₈H₅₂O₂₆Na⁺ requires 947.26390.

3-O-Acetyl-4-O-[2,3,6-tri-O-acetyl-4-O-(2,3,4,6-tetra-O-acetyl-α-D-galactopyranosyl)-β-D-galactopyranosyl]-6-O-(4-nitrophenyloxycarbonyl)-1,2-O—[(R)-1-(methoxycarbonyl)ethylidene]-α-D-glucopyranose (28). Product of debenzylation 27 (0.714 g; 0.772 mmol) and p-nitrophenyl chloroformate (0.24 mg; 1.16 mmol) were dissolved in dry DCM (3 mL) and pyridine (0.19 mL; 2.3 mmol) was added to the mixture dropwise. After 1 h, the mixture was diluted with DCM, washed with brine, concentrated and chromatographed on silica gel (hexane-acetone=2:1 to 1:1) giving pure product 28 as a white foam (82 mg; 97%). NMR (CDCl₃): δ 8.30 (d, 2H, J 3.1 Hz, C₆H₄), 7.40 (d, 2H, C₆H₄), 5.80 (d, 1H, J_{1,2} 5.2 Hz, H-1), 5.60 (dd, 1H, J_{3″,4″} 3.3 Hz, J_{4″,5″} 1.2 Hz, H-4″), 5.54 (t, 1H, J_{2,3}=J_{3,4} 2.1 Hz, H-3), 5.38 (dd, 1H, J_{2″,3″} 11.0 Hz, J_{3″,4″} 3.4 Hz, H-3″), 5.23-5.18 (m, 2H, J_{1′,2′} 7.8 Hz, J_{2′,3′} 10.7 Hz, J_{1″,2″} 3.7 Hz, H-2′, H-2″), 5.04 (d, 1H, H-1″), 4.86 (dd, 1H, J_{3′,4′} 2.8 Hz, H-3′), 4.72 (d, 1H, H-1′), 4.56-4.52 (m, 2H, J_{6a,6b} 11.3 Hz, H-6a, H-5″), 4.42-4.34 (m, 3H, H-2, H-6a′, H-6a″), 4.16 (dd, 1H, J_{5,6a} 8.4 Hz, H-6b), 4.12-406 (m, 4H, H-5, H-4′, H-6b′, H-6b″), 3.84-3.79 (m, 2H, H-4, H-5′), 3.78 (s, 3H, CH₃), 2.14 (s, 3H, CH₃), 2.13 (s, 3H, CH₃), 2.12 (s, 3H, CH₃), 2.08 (s, 3H, CH₃), 2.07 (s, 3H, CH₃), 2.06 (s, 3H, CH₃), 2.04 (s, 3H, CH₃), 2.00 (s, 3H, CH₃), 1.80 (s, 3H, CH₃). HRMS-ES m/z 1112.27015 [M+Na]⁺, C₄₅H₅₅NO₃₀Na⁺ requires 1112.27011.

3-O-Acetyl-4-O-[2,3,6-tri-O-acetyl-4-O-(2,3,4,6-tetra-O-acetyl-α-D-galactopyranosyl)-β-D-galactopyranosyl]-1,2-O—[(R)-1-(methoxycarbonyl)ethylidene]-6-O-[3-(2-methacryloylamino)propyl]carbamoyl-α-D-glucopyranose (29). Compound 28 (0.39 g; 0.36 mmol) and N-(3-aminopropyl)metacrylamide hydrochloride (83 mg; 0.47 mmol) were dissolved in dry DCM (6 mL) and triethylamine (0.2 mL) was added. The mixture was stirred at room temperature for 4 h until TLC indicated that the reaction was complete. The mixture was concentrated and chromatographed on silica gel in hexane-acetone=11:9 to provide 29 as a white foam (0.377 g; 96%). A small sample was additionally purified using HPLC C-8 column in water-methanol (product was loaded in 50% methanol and eluted from the column with 60% methanol). NMR (CDCl₃): δ 6.51-6.47 (m, 1H, NH), 5.76 (d, 1H, J_{1,2} 5.2 Hz, H-1), 5.73 (s, 1H, H₂C═C), 5.60 (dd, 1H, J_{3″,4″} 3.3 Hz, J_{4″,5″} 1.2 Hz, H-4″), 5.51 (t, 1H, J_{2,3}=J_{3,4} 2.1 Hz, H-3), 5.38 (dd, 1H, J_{2″,3″} 11.0 Hz, J_{3″,4″} 3.3 Hz, H-3″), 5.33 (s, 1H, H₂C══C), 5.31-5.29 (m, 1H, NH), 5.22-5.18 (m, 2H, J_{1′,2′} 7.8 Hz, J_{2′,3′} 10.7 Hz, J_{1″,2″} 3.7 Hz, H-2′, H-2″), 5.04 (d, 1H, H-1″), 4.84 (dd, 1H, J_{3′,4′} 2.9 Hz, H-3′), 4.65 (d, 1H, H-1′), 4.56-4.53 (m, 1H, H-5″), 4.40 (dd, 1H, J_{5′,6a′} 6.3 Hz, J_{6a′,6b′} 11.2 Hz, H-6a′), 4.33-4.35 (m, 1H, H-2), 4.27 (dd, 1H, J_{5,6a} 2.3 Hz, J_{6a,6b} 11.8 Hz, H-6a), 4.18-4.14 (m, 2H, H-6b, H-6a″), 4.12-4.07 (m 2H, J_{5′,6b′} 6.9 Hz, H-6b′, H-6b″), 4.07 (d, 1H, H-4′), 3.94-3.90 (m, 1H, H-5), 3.84 (dd, 1H, H-5′), 3.76 (s, 3H, OCH₃), 3.72-3.69 (m, 1H, H-4), 3.41-3.32 (m, 2H, NCH₂), 3.28-3.20 (m, 2H, NCH₂), 2.14 (s, 3H, CH₃), 2.12 (2 s, 6H, CH₃), 2.08 (s, 3H, CH₃), 2.07 (s, 3H, CH₃), 2.06 (s, 3H, CH₃), 2.04 (s, 3H, CH₃), 2.00 (s, 3H, CH₃), 1.97 (bs, 3H, CH₃), 1.77 (s, 3H, CH₃), 1.72-1.65 (m, 2H, CH₂). HRMS-ES m/z 1115.35409 [M+Na]⁺, C₄₆H₆₄N₂O₂₈Na⁺ requires 1115.35378.

4-O-[4-O-(α-D-galactopyranosyl)-β-D-galactopyranosyl]-1,2-O—[(R)-1-(methoxycarbonyl)ethylidene]-6-O-[3-(2-methacryloylamino)propyl]carbamoyl-α-D-glucopyranose (30). Octaacetate 29 (90 mg; 0.083 mmol) was dissolved in dry methanol (3 mL) and 1 M sodium methoxide (165 μL; 2 eq) was added. The mixture was stirred at room temperature for 3 h. The mixture was concentrated and the residue dissolved in D₂O. The solution was neutralized with CO₂ and freeze-dried to provide the product 30 as white foam (60 mg, 98%). NMR (D₂O): δ 5.64 (s, 1H, H₂C═C), 5.62 (d, 1H, J_{1,2} 4.9 Hz, H-1), 5.44 (s, 1H, H₂C═C), 4.95 (d, 1H, J_{1″,2″} 3.9 Hz, H-1″), 4.51 (d, 1H, J_{1′,2′} 7.7 Hz, H-1′), 4.41-4.35 (m 3H, H-3, H-6a, H-5″), 4.25-4.21 (dd, 1H, H-6b), 4.18-4.16 (m, 1H, H-2), 4.05-4.01 (m, 3H, H-5, H-4′, H-4″), 3.95-3.90 (m, 2H, H-5′, H-3″), 3.85-3.68 (m, 7H, J_{2′,3′} 10.2 Hz, H-4, H-3′, H-6a, H-6b, H-2″, H-6a″, H-6b″), 3.59 (dd, H-2′), 3.28 (t, 2H, J 6.9 Hz, NCH₂), 3.19 (t, 2H, J 6.7 Hz, NCH₂), 1.93 (s, 3H, CH₃), 1.78-1.72 (m, 2H, CH₂), 1.63 (s, 3H, CH₃). ES-HRMS m/z 787.23586 ([M−H+2Na]⁺); calcd for C₂₉H₄₅N₂O₂₀Na₂: 787.23556.

### Example 13

### Synthesis of a Co-Polymer of Compound 30 with HPMA (HPMA-B1)

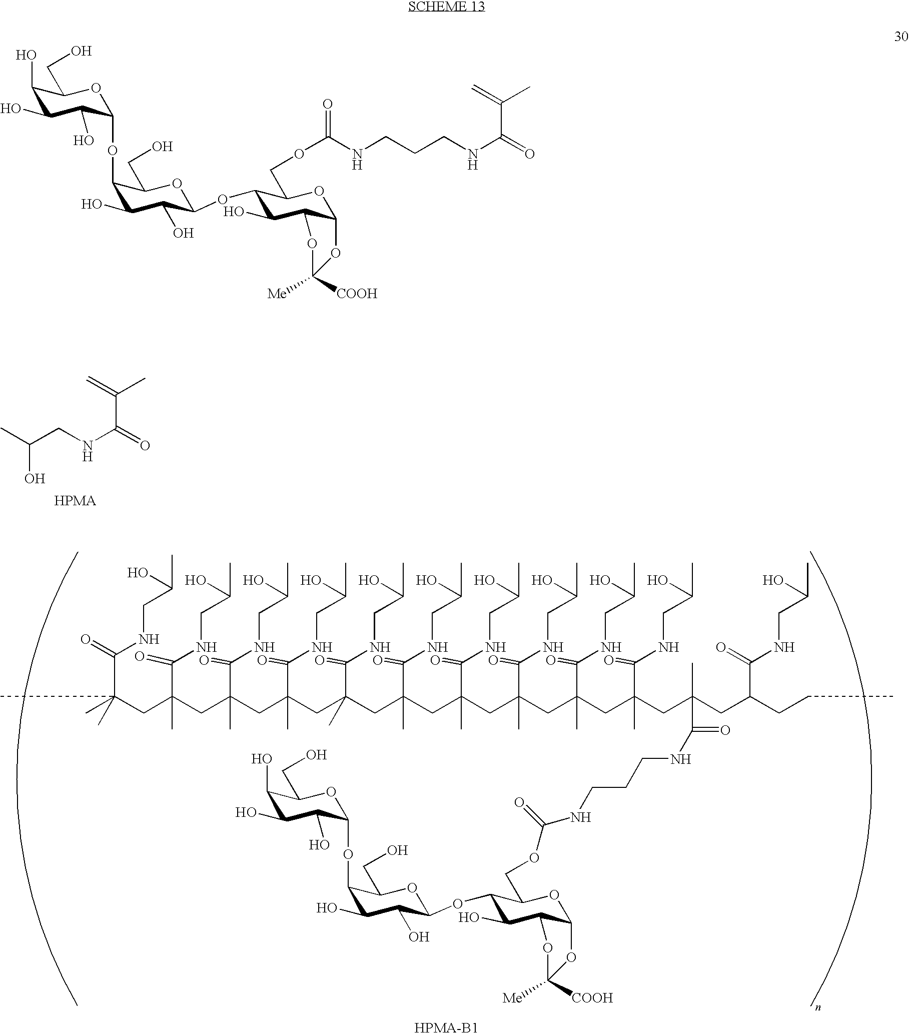

Monomer 30 (39 mg, 0.04 mmol) and HPMA monomer (101 mg; 0.7 mmol) were dissolved in degassed water (1 mL) and purged with argon. Ammonium persulfate solution (1 mg in 10 μL of degassed water) was added to the mixture followed by TEMED (1 μL). The reaction mixture was incubated at 50° C. overnight. Next day it was dialyzed against deionized water, changing water 5 times, then filtered through Milipore membrane (0.45 μm) and freeze-dried to provide the polymer as white foam (85 mg).

### Example 14

### Inhibition of Shiga Toxin 1 (Stx1) by Heterobifunctional Polymers PPM, PPI, and EPI-156

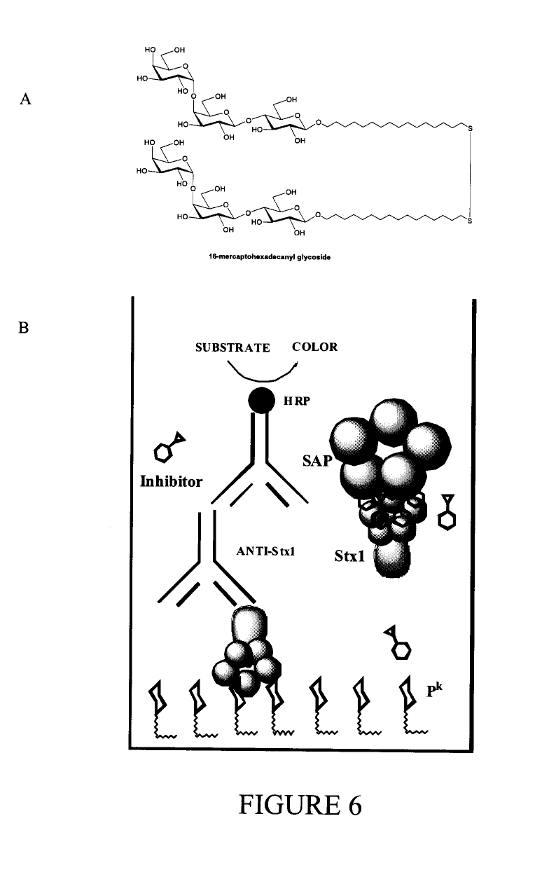

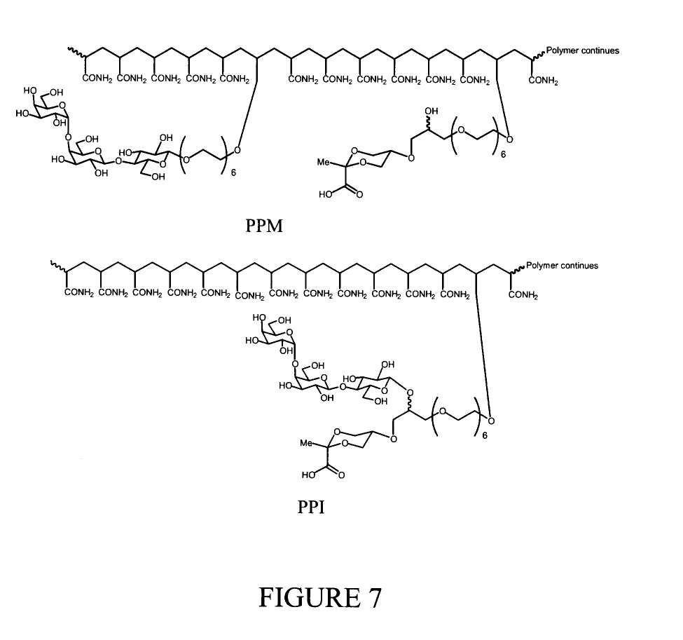

PPM, PPI and EPI-156 were assayed in the presence and absence of an endogenous protein, serum amyloid P component (SAP) for their inhibitory activity towards Shiga toxin type 1 (Stx1) in a solid-phase competitive inhibition assay (see FIGS. 6 and 7). Polystyrene ELISA plates (Maxisorp™, NUNC, Rochester, N.Y.) were coated with 10 μg/mL synthetic P^{k}-trisaccharide as a 16-mercaptohexadecanyl glycoside (Kitov P. I., et al. 1998 Carbohyd. Res. 307 (3-4):361-369) in phosphate buffered saline, pH 7.2, and incubated overnight at 4° C. All incubation volumes were 100 μL/well. HEPES buffered saline (20 mM HEPES and 0.85% NaCl, pH 7.2) supplemented with 0.05% Tween and 2.5 mM CaCl₂ (HBSTCa) was used for washing plates three times between each incubation. HBSTCa was supplemented with 0.1% bovine serum albumin (BSA) and used as a diluent in all incubations after the coating step. The inhibitors were premixed with Shiga toxin type 1 (4 ng/mL) and serum amyloid P component (20 μg/mL, Calbiochem, San Diego, Calif.) or HBSTCa+0.1% BSA (for SAP-negative format), and then the mixtures were added to the wells for 2 h incubation at RT. The toxin was detected using 1/1000 dilution of mouse ascites from an anti-Stx1 hybridoma (ATCC CRL 1794), followed by 1/2000 dilution of goat anti-mouse IgG horse radish peroxidase second antibody (Kirkegaard and Perry Laboratories, Gaithersburg, Md.). Colour was developed for 10 min with tetramethylbenzidine (Sigma, Mo.), the reaction was stopped with 1 M phosphoric acid, and the absorbance determined at a wavelength of 450 nm.

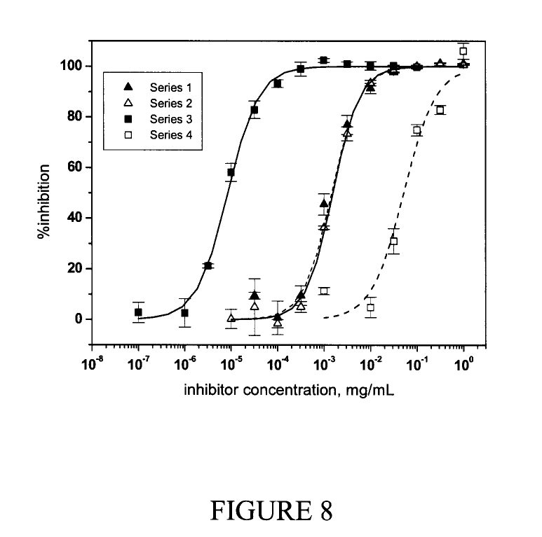

Inhibition studies demonstrated the crucial importance of pre-arrangement of the two different functionalities on the polymer scaffold. While the “pre-organized” polymer PPI shows a substantial increase (6000 times) in inhibitory activity for Stx1 in the presence of SAP, the “random” polymer PPM is almost devoid of SAP-dependent activity despite substantially higher degree of head group incorporation (see FIG. 8 and Table 1). This result: suggests that induction of a face-to-face complex is a prerequisite for activity enhancement, whereas, SAP simply brought into proximity of Stx1 (for instance by polymer PPM) is not sufficient to influence the inhibition.

TABLE 1
Comparison of inhibitory activity of polymers PPM, PPI and EPI-156
(N/A - not active at 1 g/L)

| Compound | PPM | PPI | EPI-156 |
|---|---|---|---|
| Incorporation rate, | 4.9 | 2.7 | 4 |
| mol % (P^{k}:AA ratio) | (1:19) | (1:36) | (1:24) |
| IC₅₀ (no SAP), g/L | 1.56 × 10⁻³ | 5.1 × 10⁻² | N/A |
| (M/P^{k} unit) | (4.4 × 10⁻⁷) | (1.14 × 10⁻⁵) | |
| IC₅₀ (with SAP), g/L | 1.46 × 10⁻³ | 8.43 × 10⁻⁶ | 2 × 10⁻⁶ |
| (M/P^{k} unit) | (1.47 × 10⁻⁷) | (2.4 × 10⁻⁹) | (8 × 10⁻¹⁰) |
| Amplification factor | 1.06 | 6050 | — |

The polymeric form of ligand 4 shows further improvement of SAP-dependent inhibitory power. Thus, polymer EPI-156 demonstrated excellent inhibitory activity (IC₅₀ (with SAP)=2-4 μg/L), which is not only superior to all previously reported P^{k}-containing polymers but also surpasses the inhibitory power of P^{k}-STARFISH (Kitov, P. I. et al. 2000, Nature 403:669-672), a dendrimer-type Stx antagonist, when the activity is calculated on a per-unit P^{k}-trisaccharide basis. The necessity of SAP participation in inhibition is underscored by the fact that almost no activity was detected for EPI-156 in the absence of SAP.

### Example 15

### Inhibition of Shiga Toxin 1 (Stx1) by Heterobifunctional Polymers HPMA-B1 and HPMA-B2

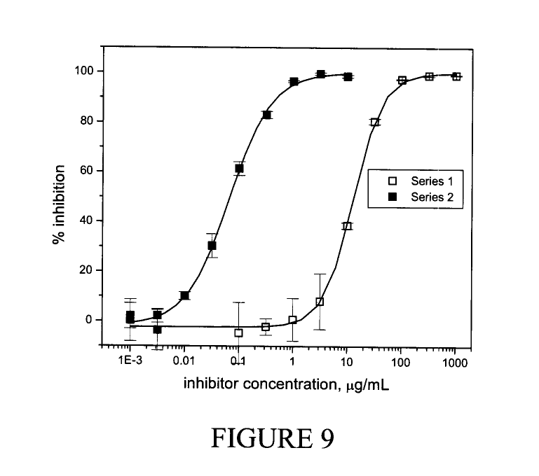

HPMA-B1 and HPMA-B2 were assayed in the presence and absence of an endogenous protein, serum amyloid P component (SAP) for their inhibitory activity towards Shiga toxin type 1 (Stx1) in a solid-phase competitive inhibition assay, as described above in EXAMPLE 14. These inhibition studies demonstrated again the crucial importance of pre-arrangement of the two different functionalities on the polymer scaffold, as well as the optimization of the pre-arrangement. HPMA-B1 and HPMA-B2 differ in the length of the linker that joins the heterobifunctional ligand to the polymeric backbone. As shown in FIG. 9, HPMA-B2 has a dramatically smaller IC₅₀ value (0.065 μg/mL) than HPMA-B1 (12.8 μg/mL), which demonstrates the importance of optimization in the pre-arrangement of the heterobifunctional ligands on the polymer.

### Example 16

### In Vitro Efficacy of EPI-156 in Vero Cell Cytotoxicity Assay

BAIT2, EPI-156 and EPI-153 were each dissolved in double-distilled, deionized water. Stock solutions of purified Stx1 and Stx2 were prepared at concentrations of 400 ng/ml and 2 mg/ml, respectively, in unsupplemented MEM. Serial dilutions, in unsupplemented MEM, of each polymer solution were prepared using a 96-well microtitre plate. Next, 5 mL of stock Stx1 or Stx2 solution was added to each well (to 80 mL final volume) of the appropriate rows in the dilution plate. The solution in each of the dilution plate wells was thoroughly mixed and the microtitre plate was incubated for 1 h at 37° C., after which 20 mL from each well was transferred to the corresponding well of a 96-well microtitre plate containing confluent Vero cell monolayers and 200 mL of MEM supplemented with fetal bovine serum. The Vero cell microtitre plate was incubated for an additional 48 h in a 37° C. incubator in an atmosphere of 5% CO₂/95% air. The Vero cell monolayers were then fixed with methanol and cytotoxicity was measured as described in Armstrong, G. D., et al., 1991 J. Infect. Dis. 164:1160-1167.

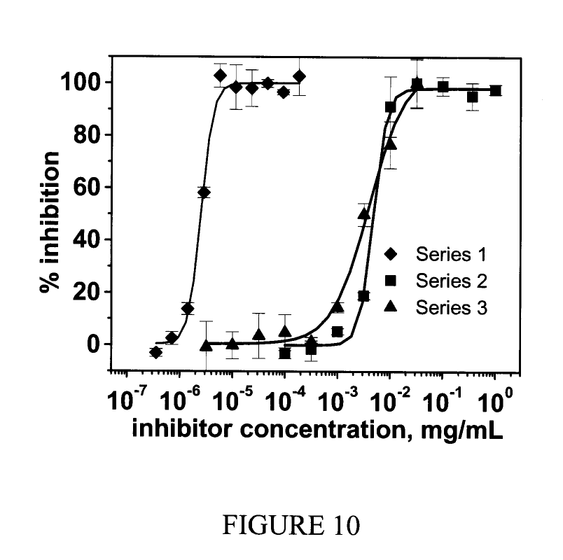

The results of Vero cytotoxicity assay are shown in FIG. 10. The heterobifunctional BAIT2 shows comparable activity to the decavalent ligand DAISY-1/8 in the presence of SAP. Furthermore, when presented on a polymeric scaffold, the activity of BAIT2 was amplified at least 3 orders of magnitude to reach the unprecedented level of 1 ng/mL for EPI-156. Radio labeling with iodine-125 did not substantially change that activity. The lactose analog, EPI-153, did not show any Vero cell protection at 3 mg/mL (data not shown).

### Example 17

### In Vivo Efficacy of EPI-156 in HuSAP Transgenic Mice

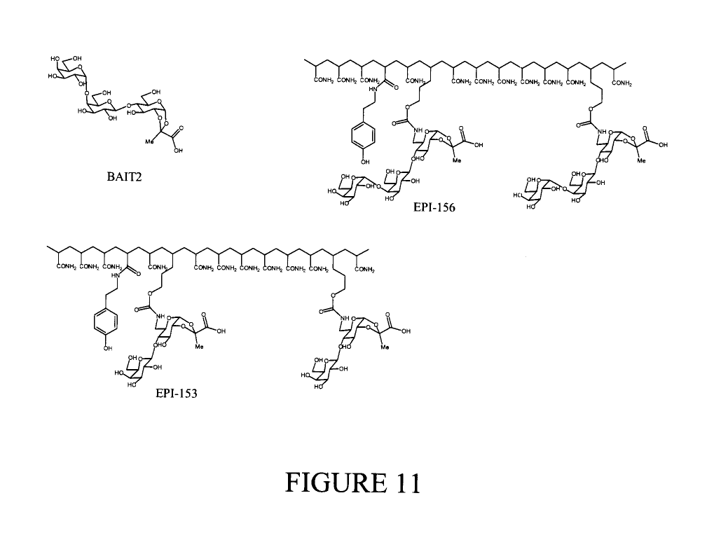

Prior art compound BAIT2 (Kitov, P. I. et al. 2008, Angew. Chemie Intl. Ed. 47:672-676) and its polymeric analog (EPI-156) were tested in vivo. The polymer EPI-153 (see FIG. 11), which contained the inactive lactose disaccharide sequence and thus did not interact with Stx1, was used as negative control, whereas, DAISY-1/8, a decavalent P^{k}-dendrimer, was a positive control since it has previously has shown in vivo protection against the Shiga like toxins, Stx1 and Stx2 (Mulvey, G. L. 2003, J. Infec. Dis. 187:640-649).

The mouse intoxication model (Armstrong, G. D., et al., 2006, J. Infect. Dis. 193:1220-1124) measures mouse survival following administration of Stx1 and various inhibitory polymers of the prior art and of the present invention. HuSAP mice were injected intravenously via the tail vein with Stx1 (20 ng/g) and they were monitored every 4 hours for signs of shigatoxemia. Mice displaying signs of shigatoxemia were euthanized.

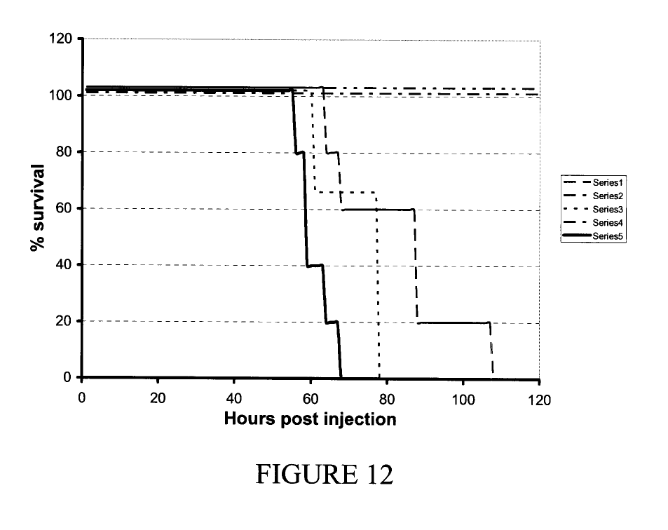

As seen in FIG. 12, series 1 represents the percentage of mouse survival following administration of DAISY 1/8 at 0.5 mg/mouse. Series 2 represents the percentage of mouse survival following administration of EPI-156 at 50 μg/mouse. Series 3 represents the percentage of mouse survival following administration of EPI-153 at 0.6 mg/mouse and HuSAP at 600 μg/mouse. Series 4 represents the percentage of mouse survival following administration of EPI-156 at 50 μg/mouse and HuSAP at 600 μg/mouse. Series 5 represents the percentage of mouse survival following administration of BAIT2 at 4 mg/mouse.

All HuSAP transgenic mice were saved from lethal injection of 20 ng/g Stx1 when co-administered with EPI-156 at only 50 μg/mouse. Of all other tested compounds, only DAISY-1/8 demonstrated some efficacy by delaying onset of symptoms in several mice, for which 10 times higher amount of DAISY-1/8 was required. Univalent ligand BAIT2 failed to show effect even at 50 times higher concentration.

### Example 18

### Organ Localization of Labeled EPI-156

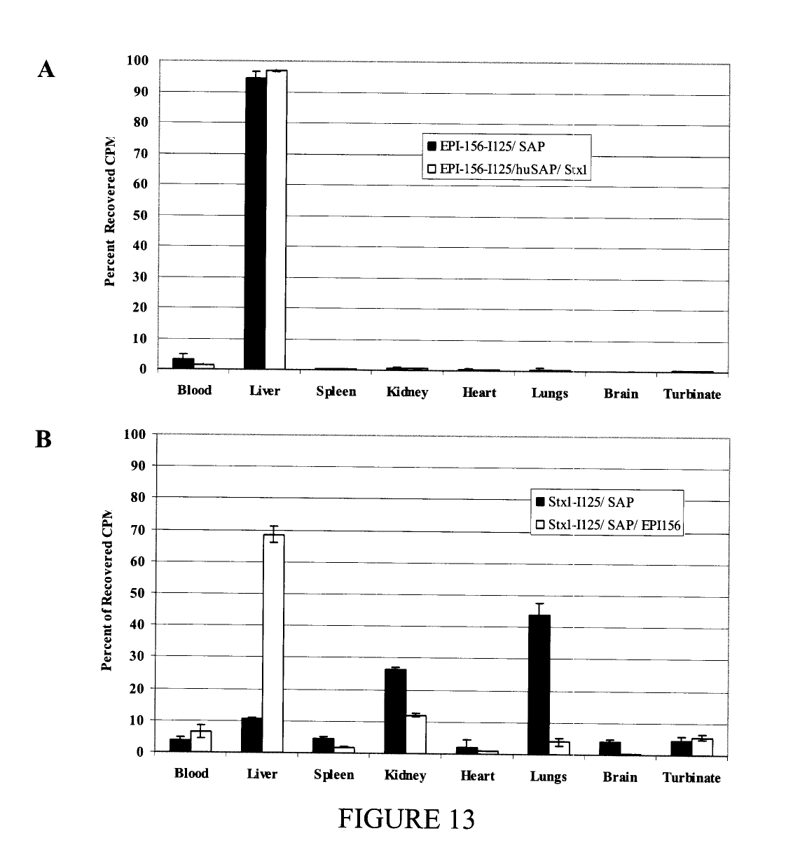

For these studies, EPI-156 was modified by the addition of a tyrosine residue to allow for iodination (see FIG. 11). Organ localization of radioactively labeled EPI-156 (EPI-156-¹²⁵I) and Shiga toxin (Stx1-¹²⁵I) were determined following post-intravenous injection into transgenic mice expressing human SAP (HuSAP mice) (Zhao, X. et al., 1992, Journal of Biochemistry 111:736-738). HuSAP mice received 900 ng of EPI-156-¹²⁵I (1.14×10⁷ CPM/μg) via intravenous tail vein injection and were euthanized at 4 hours. Following euthanization, radioactivity was counted in different organs. FIG. 13A shows the results from this assay. Solid bars represent the organ distribution of a mixture of EPI-156-¹²⁵I and HuSAP, whereas open bars represent the organ distribution of a mixture of EPI-156-¹²⁵I, HuSAP and Stx1. From FIG. 13A, it can be observed that the heterobifunctional polymeric ligand is directed to the liver. In fact, greater than 95% of EPI-156-¹²⁵I localized exclusively in the liver. Co-injection of Stx1 only slightly increased the amount of the polymer found in the liver after 4 h. In FIG. 13B, mice received 20 ng/g of Stx1-¹²⁵ (4.81×10⁶ CPM/μg) via tail vein injection. Solid bars represent the organ distribution of a mixture of Stx1 and HuSAP, whereas open bars represent the organ distribution of a mixture of Stx1, HuSAP and non-labeled EPI-156. From FIG. 13B, it can be observed that, in the presence of multivalent heterobifunctional polymers of the present invention, the toxin Stx1 is directed to the liver instead of being directed to the kidneys and lungs, which is observed in the absence of the polymers.

When the ¹²⁵I-labeled Stx1 was injected in mice transgenic for human HuSAP, only 10% of the radioactivity was found in the liver (see FIG. 13B). Most of the toxin was localized in lungs and kidney and even in brain (5%). The co-injection of heterobifunctional polymer EPI-156 diverted almost 70% of toxin from these vital organs to the liver (see FIG. 13B).

The ability of heterobifunctional polymer EPI-156 to alter organ distribution of injected toxin may explain the 100% survival of mice treated with EPI-156 in the mouse intoxication model (see FIG. 12).

1. A multivalent heterobifunctional polymer for binding to a biological target exhibiting biological activity and to an effector template which can affect the biological activity of the biological target or detect the presence of the biological target, the polymer comprising a plurality of pre-arranged heterobifunctional ligands connected thereto, the heterobifunctional ligands comprising:
(a) a first functionality capable of binding to the biological target, and
(b) a second functionality capable of binding to the effector template,
wherein the heterobifunctional ligands are pre-arranged on the polymer so as to form a ternary complex between the polymer, the biological target and the effector template.

2. The multivalent heterobifunctional polymer of claim 1, wherein the first functionality and the second functionality can be selected from the group consisting of an amino acid, a peptide, a derivatized peptide, a monosaccharide, an oligosaccharide, a vitamin, a nucleotide, a nucleotide analog, a polynucleotide, a polynucleotide analog, a cell nutrient, an antigenic determinant, a small drug-like compound, a hapten, an antibody or antibody fragment, a cell surface receptor, and combinations and analogs thereof.

3. The multivalent heterobifunctional polymer of claim 1, wherein the biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, an archaebacteria, a fungus, a viral particle, a bacterial toxin, viral lectins, a cancer cell, B cells, and combinations and analogs thereof.

4. The multivalent heterobifunctional polymer of claim 1, wherein the effector template can be selected from the group consisting of a multivalent receptor, a multivalent protein, a protein, a peptide, a derivatized peptide, an antibody, a membrane-bound receptor, and combinations and analogs thereof.

5. The multivalent heterobifunctional polymer of claim 1, wherein the first functionality and the second functionality are attached to a common atom, wherein the common atom is attached directly or via a linker to or into the polymer backbone.

6. The multivalent heterobifunctional polymer of claim 1, wherein the first functionality and the second functionality are directly, or via an optional linker, attached to one another, and either the first functionality or the second functionality is attached directly or via a linker to or into the polymer backbone.

7. The multivalent heterobifunctional polymer of claim 1, wherein the first functionality binds a multivalent biological target.

8. The multivalent heterobifunctional polymer of claim 7, wherein the first functionality binds a bacterial toxin.

9. The multivalent heterobifunctional polymer of claim 8, wherein the toxin is selected from the group consisting of Shiga toxins, heat-labile enterotoxin, subtilase cytotoxin, and cholera toxin.

10. The multivalent heterobifunctional polymer of claim 9, wherein the first functionality is a trisaccharide.

11. (canceled)

12. The multivalent heterobifunctional polymer of claim 7, wherein the first functionality binds a viral particle.

13-15. (canceled)

16. The multivalent heterobifunctional polymer of claim 1, wherein the first functionality binds a cell surface receptor of a cancer cell.

17-22. (canceled)

23. The multivalent heterobifunctional polymer of claim 1, wherein the second functionality binds SAP.

24. The multivalent heterobifunctional polymer of claim 23, wherein the second functionality is a cyclic pyruvate ketal.

25. The multivalent heterobifunctional polymer of claim 1, wherein the second functionality binds an antibody.

26-27. (canceled)

28. The multivalent heterobifunctional polymer of claim 1, wherein the polymer is selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers.

29. A multivalent heterobifunctional polymer for binding to a biological target exhibiting biological activity and to an effector template which can affect the biological activity of the biological target or detect the presence of the biological target, the polymer having the formula: 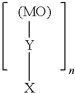
wherein “X” represents a polymeric backbone of the multivalent polymer; “MO” represents a heterobifunctional ligand, wherein “M” represents a first functionality capable of binding to the biological target and “O” represents a second functionality capable of binding to the effector template;
“Y” represents an optional linker that connects “MO” to or into the polymeric backbone; and
“n” represents an integer selected such that a sufficient number of heterobifunctional ligands are presented in the polymer for an intended use.

30. The multivalent heterobifunctional polymer of claim 29, wherein “n” is selected such that the number of heterobifunctional ligands on the polymer is the same as or greater than the number of receptors on the biological target and the effector template.

31. The multivalent heterobifunctional polymer of claim 29, wherein the first functionality binds a multivalent biological target.

32. The multivalent heterobifunctional polymer of claim 31, wherein the first functionality binds a bacterial toxin.

33. (canceled)

34. The multivalent heterobifunctional polymer of claim 31, wherein the first functionality is a trisaccharide.

35. (canceled)

36. The multivalent heterobifunctional polymer of claim 31, wherein the first functionality binds a viral particle.

37-39. (canceled)

40. The multivalent heterobifunctional polymer of claim 29, wherein the first functionality binds a cell surface receptor of a cancer cell.

41-46. (canceled)

47. The multivalent heterobifunctional polymer of claim 29, wherein the second functionality binds SAP.

48. The multivalent heterobifunctional polymer of claim 47, wherein the second functionality is a cyclic pyruvate ketal.

49. The multivalent heterobifunctional polymer of claim 29, wherein the second functionality binds an antibody.

50-51. (canceled)

52. The multivalent heterobifunctional polymer of claim 29, wherein the polymer is selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers.

53. The multivalent heterobifunctional polymer of claim 29, wherein “M” is connected to or into the polymeric backbone.

54. The multivalent heterobifunctional polymer of claim 29, wherein “M” is connected to or into the polymeric backbone through linker “Y”.

55. The multivalent heterobifunctional polymer of claim 29, wherein “O” is connected to or into the polymeric backbone.

56. The multivalent heterobifunctional polymer of claim 55, wherein “O” is connected to or into the polymeric backbone through linker “Y”.

57. The multivalent heterobifunctional polymer of claim 29, wherein “M” and “O” are connected to each other by a linker.

58. The multivalent heterobifunctional polymer of claim 29, wherein the first functionality and the second functionality can be selected from the group consisting of an amino acid, a peptide, a derivatized peptide, a monosaccharide, an oligosaccharide, a vitamin, a nucleotide, a nucleotide analog, a polynucleotide, a polynucleotide analog, a cell nutrient, an antigenic determinant, a small drug-like compound, a hapten, an antibody or antibody fragment, a cell surface receptor, and combinations and analogs thereof.

59. The multivalent heterobifunctional polymer of claim 29, wherein the biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, an archaebacteria, a fungus, a viral particle, a bacterial toxin, viral lectins, a cancer cell, B cells, and combinations and analogs thereof.

60. The multivalent heterobifunctional polymer of claim 29, wherein the effector template can be selected from the group consisting of a multivalent receptor, a multivalent protein, a protein, a peptide, a derivatized peptide, an antibody, a membrane-bound receptor, and combinations and analogs thereof.

61. A multivalent heterobifunctional polymer for binding to a biological target exhibiting biological activity and to an effector template which can affect the biological activity of the biological target or detect the presence of the biological target, the polymer having the formula: 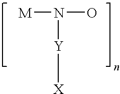
wherein “X” represents a polymeric backbone of the multivalent polymer;
“M-N-O” represents a heterobifunctional ligand, wherein “M” represents a first functionality capable of binding to the biological target, “O” represents a second functionality capable of binding to the effector template, and “N” represents a linker connecting “M” and “N”;
“Y” represents an optional linker that connects the heterobifunctional ligand to or into the polymeric backbone; and
“n” represents an integer selected such that a sufficient number of heterobifunctional ligands are presented in the polymer for an intended use.

62. The multivalent heterobifunctional polymer of claim 61, wherein “n” is selected such that the number of heterobifunctional ligands on the polymer is the same as the number of receptors on the biological target and the effector template.

63. The multivalent heterobifunctional polymer of claim 61, wherein the first functionality binds a multivalent biological target.

64. The multivalent heterobifunctional polymer of claim 61, wherein the first functionality binds a bacterial toxin.

65. The multivalent heterobifunctional polymer of claim 64, wherein the toxin is selected from the group consisting of Shiga toxins, heat-labile enterotoxin, subtilase cytotoxin, and cholera toxin.

66. The multivalent heterobifunctional polymer of claim 65, wherein the first functionality is a trisaccharide.

67. (canceled)

68. The multivalent heterobifunctional polymer of claim 61, wherein the first functionality binds a viral particle.

69-70. (canceled)

71. The multivalent heterobifunctional polymer of claim 68, wherein the first functionality binds a viral lectin.

72. The multivalent heterobifunctional polymer of claim 61, wherein the first functionality binds a cell surface receptor of a cancer cell.

73-78. (canceled)

79. The multivalent heterobifunctional polymer of claim 61, wherein the second functionality binds SAP.

80. The multivalent heterobifunctional polymer of claim 61, wherein the second functionality is a cyclic pyruvate ketal.

81. The multivalent heterobifunctional polymer of claim 61, wherein the second functionality binds an antibody.

82-83. (canceled)

84. The multivalent heterobifunctional polymer of claim 61, wherein the polymer is selected from the group consisting of polyacrylamide, poly[N-(2-hydroxypropyl)methacrylamide], polysaccharide, dextran, glycosaminoglycan, hyaluronic acid, poly(amino acid), poly(aspartic acid), poly(glutamic acid), combinations thereof, and other pharmaceutically acceptable polymers.

85. The multivalent heterobifunctional polymer of claim 61, wherein the first functionality and the second functionality can be selected from the group consisting of an amino acid, a peptide, a derivatized peptide, a monosaccharide, an oligosaccharide, a vitamin, a nucleotide, a nucleotide analog, a polynucleotide, a polynucleotide analog, a cell nutrient, an antigenic determinant, a small drug-like compound, a hapten, an antibody or antibody fragment, a cell surface receptor, and combinations and analogs thereof.

86. The multivalent heterobifunctional polymer of claim 61, wherein the biological target can be selected from the group consisting of a multivalent receptor, a multivalent protein, a protein, a peptide, a derivatized peptide, an antibody, a membrane-bound receptor, a bacteria, a Gram-positive bacteria, a Gram-negative bacteria, a unicellular parasite, an archaebacteria, a fungus, a viral particle, a bacterial toxin, viral lectins, a cancer cell, B cells, and combinations and analogs thereof.

87. The multivalent heterobifunctional polymer of claim 61, wherein the effector template can be selected from the group consisting of a multivalent receptor, a multivalent protein, a protein, a peptide, a derivatized peptide, an antibody, a membrane-bound receptor, and combinations and analogs thereof.

88-99. (canceled)

100. A pharmaceutical composition for affecting the biological activity of a biological target in a biological system, the composition comprising:
(a) a multivalent heterobifunctional polymer for binding to the biological target exhibiting biological activity and to an effector template which can affect the biological activity of the biological target, the polymer comprising a plurality of pre-arranged heterobifunctional ligands, the heterobifunctional ligands comprising a first functionality capable of binding to the biological target, and a second functionality capable of binding to the effector template, wherein the heterobifunctional ligands are pre-arranged on the polymer so as to form a ternary complex between the polymer, the biological target and the effector template; and
(b) a pharmaceutically acceptable excipient.

101-113. (canceled)
